(19) United States
(12) Patent Application Publication
(10) Pub. No.: US 2021/0388418 A1
(43) Pub. Date: Dec. 16, 2021

(54) Method for Quantifying Molecular Activity in Cancer Cells of a Human Tumour

(71) Applicant: AGENCY FOR SCIENCE, TECHNOLOGY AND RESEARCH, Singapore (SG)

(72) Inventors: Anders Skanderup, Singapore (SG); Umesh Ghoshdastider, Singapore (SG)

(21) Appl. No.: 17/309,055

(22) PCT Filed: Oct. 18, 2019
(86) PCT No.: PCT/SG2019/050517

§ 371 Date: Apr. 16, 2021

(30) Foreign Application Priority Data
Oct. 18, 2018 (SG) 10201809232S

(51) Int. Cl. C12Q 1/6809; G16B 25/10

(52) U.S. Cl. CPC C12Q 1/6809; G16B 25/10

(57) ABSTRACT

Disclosed herein is a method for predicting expression profiles of cancerous and non-cancerous cells respectively based on multiple sets of expression profiles, wherein each set of the multiple sets of expression profiles is obtained from tumour-derived samples comprising a mixture of cancerous and non-cancerous cells of one tumour type.

### CROSS-REFERENCE TO RELATED APPLICATIONS

This application claims the benefit of priority of Singapore provisional application no. 10201809232S, filed 18 Oct. 2018, the contents of it being hereby incorporated by reference in its entirety for all purposes.

### FIELD OF THE INVENTION

The present invention relates generally to the field of bioinformatics. In particular, the present invention relates to identifying biomarkers for use in the detection and diagnosis of cancer.

### BACKGROUND OF THE INVENTION

Tumours are heterogeneous masses of malignant mutated cancer cells, non-malignant (stromal and immune) cells, as well as intercellular connective structures. Collectively, these components form the tumour microenvironment (TME), which is a multi-faceted cellular environment that both constrains and supports the evolving tumour. Understanding how cancer cells interact with their environment inside human tumours is a long-standing challenge. Importantly, cancer cells usually comprise <60% of all cells in the combined tumour mass. When profiling molecular activity (i.e. mRNA expression) in bulk tumour samples it is impossible to determine if a given factor is expressed predominantly in cancer or non-cancer cells. Any molecular readout will be a sum of signals coming from the cancer and the many non-cancer cells in the TME.

Experimental models can simulate and measure crosstalk in the tumour microenvironment, but such models are generally limited by how tumour cells rapidly adapt physiology outside their natural environment. Immunohistochemistry (IHC) can directly measure chosen proteins in tumour tissue, but is not suited for large-scale and unbiased discovery. It can be performed on a single tumour, but is labour intensive, biased (as it can only be applied for selected markers), and is not quantitative (based on a percentage of cells expressing marker). Also, current bulk tumour transcriptome sequencing does not inform specifically about cancer cells. Instead, transcriptome-wide profiles of cancer and stromal cell may be generated using micro-dissection or single-cell profiling of tumour tissue, but these approaches are difficult to apply to tumour biopsies and disassociation may to some extent also confound cell physiology and gene expression profiles. Furthermore, above methods cannot be applied retrospectively to existing large-scale cancer genomics bulk tumour data, representing a vast and mostly unexplored resource for studying cross-talk in the tumour microenvironment.

One major branch of oncology drug development is focusing on development of antibodies (or antibody-conjugated-drugs) that specifically target antigens/proteins inside or on the surfaces of cancer cells. It is therefore critical in the early phases of drug development to have access to accurate molecular profiles of cancer cells. While experimental models (cell lines and animal models) can provide an approximation, such models are generally limited by how cancer cells rapidly adapt physiology outside their natural environment. For example, EGFR expression (and EGFR gene copies) in glioblastoma cancer cells is greatly reduced immediately upon culturing of the cancer cells in vitro.

Cancer cell gene expression can currently also be estimated with single cell profiling or laser micro-dissection. However, these approaches have limitations: the molecular profiles are biased after cell disassociation, the techniques require lots of work and are expensive, they cannot easily separate, for example, non-malignant from malignant (cancer) epithelial cells, and they cannot readily be applied to standard frozen or Formalin-Fixed Paraffin-Embedded (FFPE) tumour samples, nor are these methods scalable.

There is therefore a need for technologies that allow high-throughput profiling of cancer cells ex vivo. Furthermore, other desirable features and characteristics will become apparent from the subsequent detailed description and the appended claims, taken in conjunction with the accompanying drawings and this background of the disclosure.

### SUMMARY OF THE INVENTION

In one aspect the present invention refers to a method of predicting expression profiles of cancerous and non-cancerous cells, respectively, based on multiple sets of expression profiles, wherein each set of the multiple sets of expression profiles is obtained from tumour-derived samples comprising a mixture of cancerous and non-cancerous cells of one tumour type, wherein the method comprises: a. determining tumour purity values for the one or more tumour-derived samples; b. providing different sets of expression profiles, wherein the sets of expression profiles comprise combined expression data for multiple or all molecules expressed by cancerous and non-cancerous cells comprised in the one or more tumour-derived samples; c. deconvoluting each combined expression data referred under b. by extrapolating expression profiles of the multiple or all molecules expressed in the different tumour samples with different tumour purity values to a tumour purity value at least substantially equal to 1 or 0; thereby predicting the expression profiles of the cancerous and non-cancerous cells respectively from the sets of expression profiles.

### BRIEF DESCRIPTION OF THE DRAWINGS

The invention will be better understood with reference to the detailed description when considered in conjunction with the non-limiting examples and the accompanying drawings, in which:

FIG. 1 shows an illustration comparing conventional clinical sequencing and TUMERIC-solo sequencing in accordance with a present embodiment.

FIG. 2 shows an overview illustration of the TUMERIC sequencing process in accordance with the present embodiment.

FIG. 3 shows a flow diagram of the overall TUMERIC-solo process in accordance with the present embodiment.

FIG. 4 shows a flow diagram of the TUMERIC-solo tumour purity estimation process of FIG. 3 in accordance with the present embodiment.

FIG. 5 shows a flow diagram of the TUMERIC-solo transcriptome deconvolution of FIG. 3 in accordance with the present embodiment.

FIG. 6 shows a working example of tumour transcriptome deconvolution in accordance with the present embodiment wherein FIG. 6a shows estimated tumour purity values for around 8000 bulk tumour samples across 20 solid tumour types; FIG. 6b shows genes specifically expressed in cancer and stromal cells across cancer types; as expected, only cancer-cell specific genes are affected by DNA copy number alterations in the corresponding tumours; FIG. 6c shows the inferred cancer and stroma compartment expression levels for 280 known stromal-specific genes; FIG. 6d shows the inferred cancer and stroma compartment expression levels in melanoma (skin cutaneous melanoma—SKCM), as well as bulk tumour measurements, for cancer and stroma specific genes previously identified with melanoma tumour single cell RNA sequencing (scRNA-sequencing); FIG. 6e shows genes and pathways which are ordered by inferred expression difference between cancer and stroma compartments in each tumour type; FIG. 6f shows protein expression inferred for cancer and stroma compartments in (OV) and breast (BRCA) cancer cohorts using iTRAQ protein quantification data and compared to RNA sequencing data from the same tumors; and FIG. 6g shows genes with highly variable cancer vs. stroma mRNA expression differences across cancer types as identified, where immunohistochemistry (IHC) staining data was compared to deconvoluted RNA sequencing data for the gene (S100A6) with highest mRNA abundance.

FIG. 7 shows the results of the inference of crosstalk between cancer and stromal cells in accordance with the present embodiment, wherein FIG. 7a depicts a Relative Crosstalk (RC) score which estimates a relative flow of signalling in four possible directions between cancer and stromal cell compartments, including a bulk (non-deconvoluted) normal tissue signalling estimate; FIG. 7b depicts a median RC score across twenty solid tumour types estimated and plotted for each direction of signalling; FIGS. 7c and 7d displays five ligand-receptor pairs with highest median autocrine cancer signalling score (FIG. 7c) and highest median paracrine stroma to cancer signalling score (FIG. 7d) across tumour types, RC scores for individual pairs and cancer types; FIG. 7e depicts RC scores for canonical EGF-family ligand-receptor pairs across breast cancer subtypes; and FIGS. 7f and 7g depict estimated expression of EGF-family receptors (f) and ligands (g) in cancer and stromal cell compartments across the breast cancer subtypes, normal tissue non-deconvoluted expressions being included for comparison.

FIG. 8 shows an example query to illustrate the process of identifying membrane protein drug targets in glioblastoma tumors using TUMERIC. In this query, the user specifies the tumor type (Glioblastoma) and further specifies a genetic/molecular subtype of tumors to analyse (here tumors without IDH1 mutations). Known membrane proteins are then ranked by their overall bulk tumor expression (x-axis) and the extent, as inferred by TUMERIC, that they are expressed specifically in cancer cells (y-axis). Predicted toxicity of each target, e.g. derived from gene expression in healthy vital organs such as brain/heart/kidney, can be co-visualized and aid in the target selection process.

FIG. 9 shows a schematic illustration representing an outline of tumour transcriptome deconvolution methodologies and platforms in accordance with the present embodiment wherein FIG. 9A depicts a concept of the algorithm for tumour transcriptome deconvolution in accordance with the present embodiment utilized for inferring cancer-cell specific drug targets. FIG. 9B depicts an overview of components needed for such a platform: a large data warehouse of bulk patient tumor samples with genomic and transcriptomic data, fast algorithms (online transcriptome deconvolution) and visualization to facilitate exploration and identification of drug targets and biomarkers, and an example query to illustrate the process of identifying drug targets in glioblastoma tumors.

FIG. 10 shows data that TUMERIC-Solo can estimate cancer and stromal-cell expression of PD-L1 in an individual lung cancer patient (A014). TUMERIC-Solo gene expression deconvolution of PD-L1 in a single lung cancer patient (A014) in accordance with the present embodiment as compared to data PD-L1 expression inferred from a cohort of patients (global, TUMERIC applied to about 60 lung cancer patients) in accordance with the present embodiment; measured bulk tumor gene expression included for comparison.

FIG. 11 shows data from TUMERIC-Solo applied to a single lung cancer patient (A014) in accordance with the present embodiment as compared to data from a cohort of patients (global, TUMERIC applied to about 60 lung cancer patients) in accordance with the present embodiment. Deconvoluted cancer and stromal-cell gene expression of four genes shows concordance of single patient TUMERIC-solo and multi-patient TUMERIC (global); measured bulk tumor gene expression included for comparison.

FIG. 12 shows detailed data from TUMERIC-Solo applied to sectors of a single lung cancer patient tumor (A014) in accordance with the present embodiment as compared to data from a cohort of patients (TUMERIC applied to about 60 lung cancer patients) in accordance with the present embodiment. The plots show the association of measured bulk gene expression (y-axis) with estimated tumor purity (x-axis) for three selected genes.

FIG. 13 shows TUMERIC-Solo applied to a set of published biomarker genes associated with response to Pembrolizumab treatment response. The expression of the 6 genes in cancer and stromal cells of a single lung cancer patient (A014) was determined with TUMERIC-solo and compared to data from a cohort of patients (TUMERIC applied to about 60 lung cancer patients). Measured bulk tumor gene expression included for comparison.

FIG. 14 shows the relative change (signal-to-noise) in gene expression of 6 Pembrolizumab biomarker genes when evaluated using bulk or TUMERIC-solo deconvoluted gene expression for a lung cancer patient (A014). The change in gene expression is measured relative to bulk, cancer, and stromal cell expression determined using data from a cohort of patients (TUMERIC applied to about 60 lung cancer patients). The expression of PD-L1/CD274 is compared for cancer cells, whereas expression of the other 5 biomarkers are compared for stromal cells.

FIG. 15 shows graphs depicting patient specific recommendation of therapeutic antibodies with TUMERIC-Solo (left) in accordance with the present embodiment as compared to a similar recommendation based on measured bulk gene expression (right). The graphs show absolute (x-axis) and relative (y-axis, vs. normal lung tissue) expression of known membrane proteins in a lung cancer patient (A014). Based on the data shown, one would nominate a CLDN6 antibody treatment (Antibody or Antibody-Drug Conjugate) for this lung cancer patient.

FIG. 16 shows TUMERIC used to identify biomarkers associated with response to Pembrolizumab treatment in gastric cancer. TUMERIC analysis identified genes with robust cancer or stromal cell gene expression dysregulation in tumors of responders (R) as compared to non-responders (PD). The signal-to-noise ratio (R vs. PD) measured with TUMERIC (y-axis) is shown together with the signal-to-noise ratio measured with naïve bulk gene expression profiling (x-axis).

FIG. 17 shows data for Biglycan (BGN) expression in patients with different responses (responder, R; stable disease, SD; progressive disease, PD) to pembrolizumab treatment. Bulk tumor gene expression of BGN (left) as compared to TUMERIC deconvoluted gene expression of BGN in cancer cells (right): BGN is highly overexpressed in cancer cells of non-responders (PD) with only a modest change measurable with bulk gene expression.

FIG. 20 shows detailed data from TUMERIC applied to Biglycan (BGN) expression in patients with different responses (responder, R; stable disease, SD; progressive disease, PD) to pembrolizumab treatment. The plots show the association of measured BGN bulk gene expression (y-axis) with estimated tumor sample purity (x-axis) for the three treatment response groups.

FIG. 19 shows an overview illustration of the TUMERIC-solo sequencing process in accordance with the present embodiment.

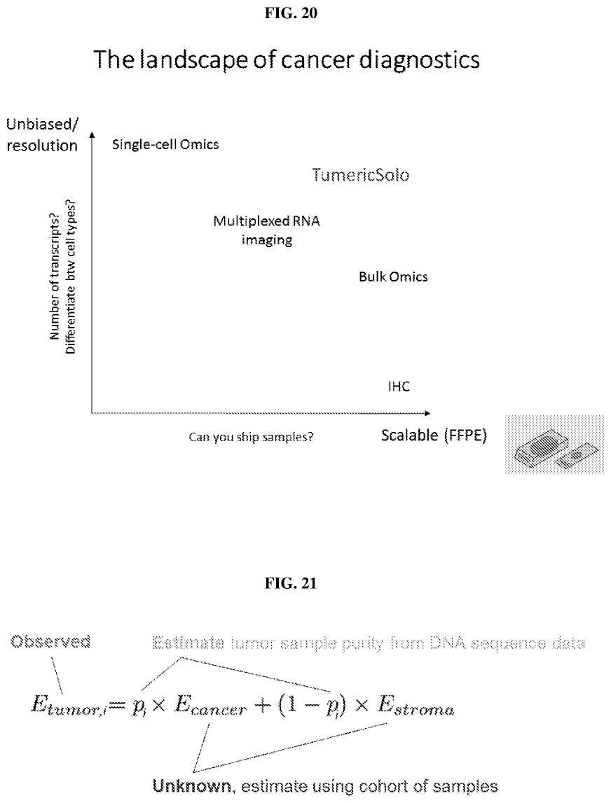

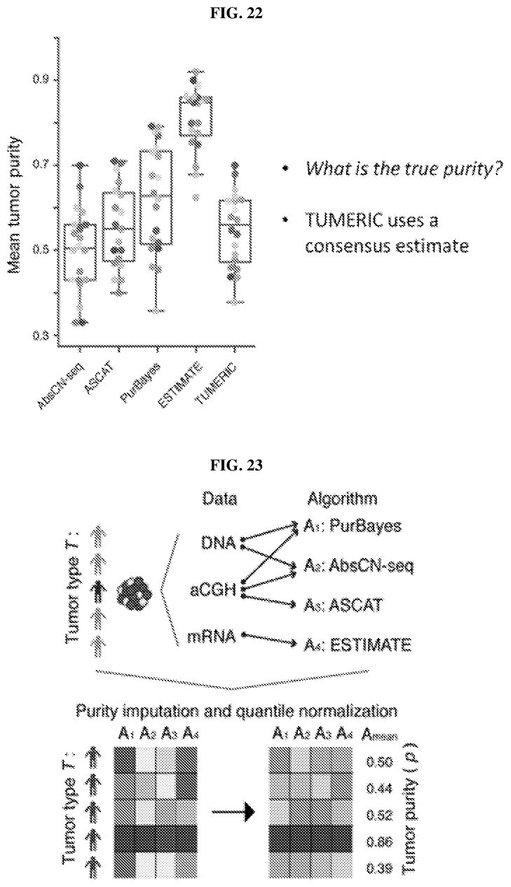

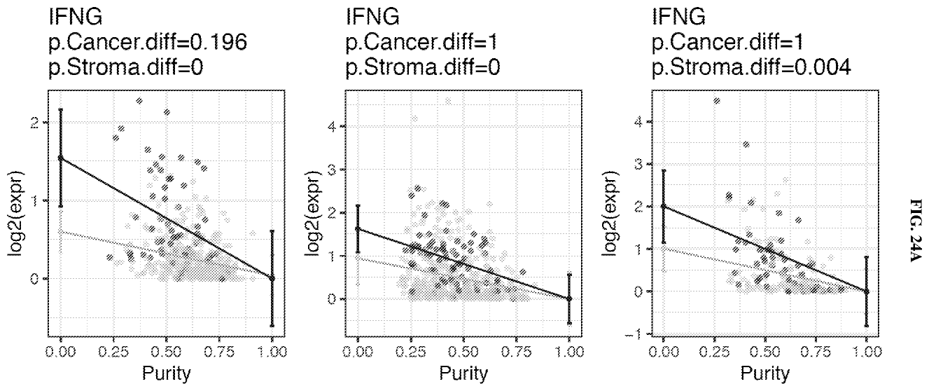

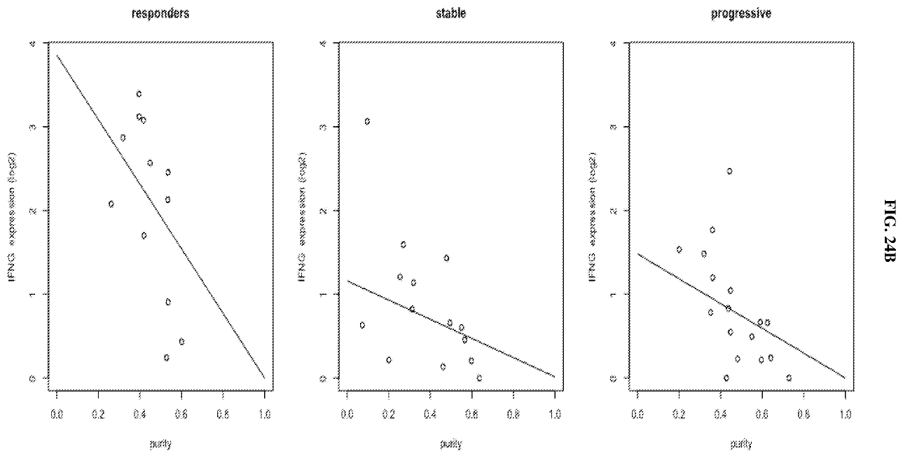

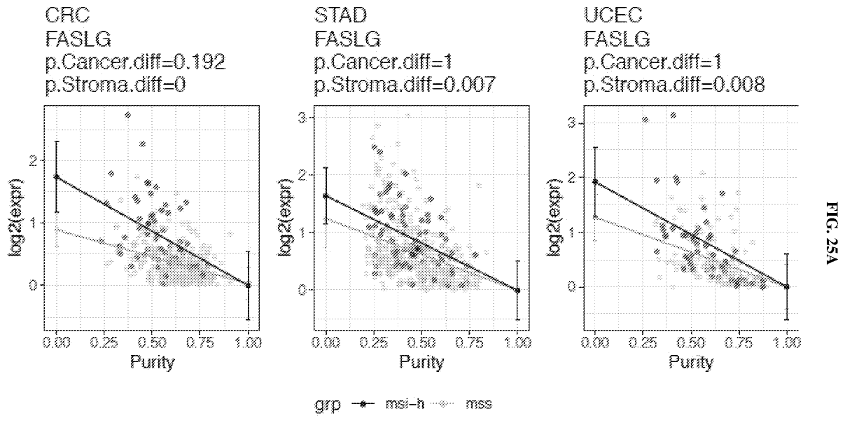

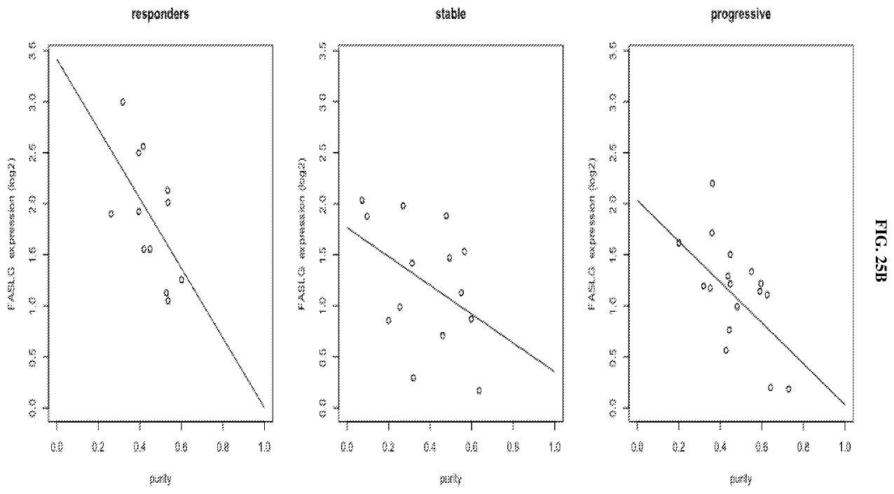

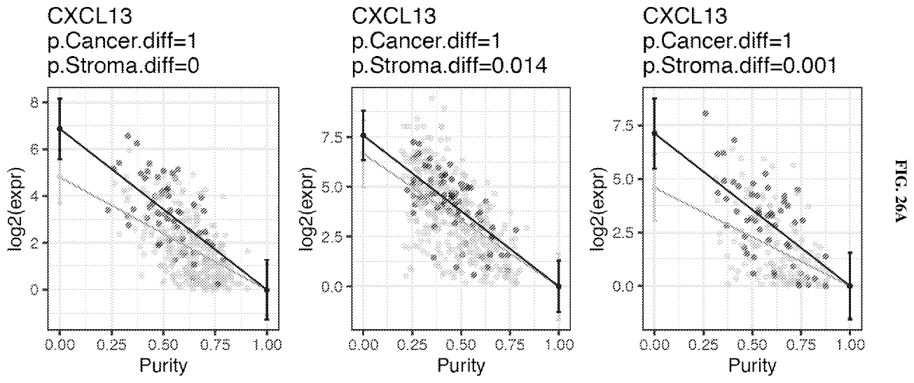

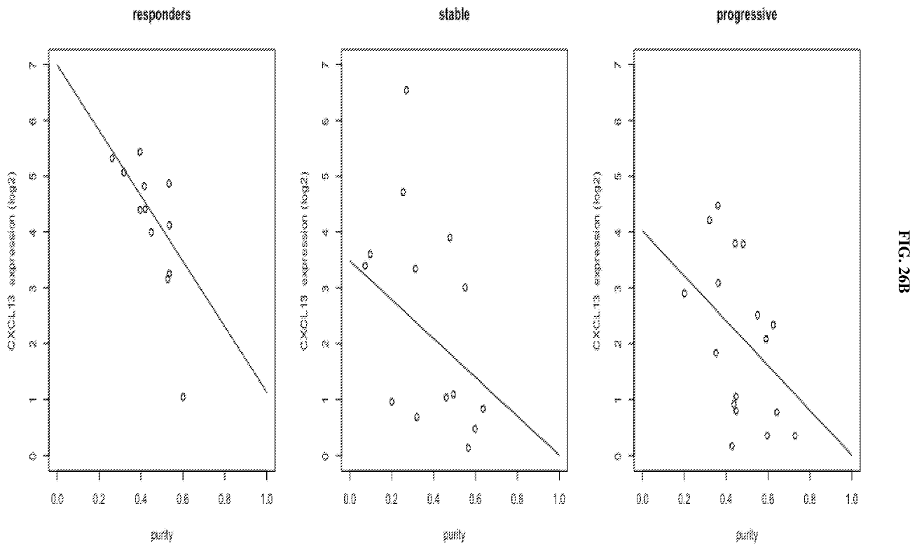

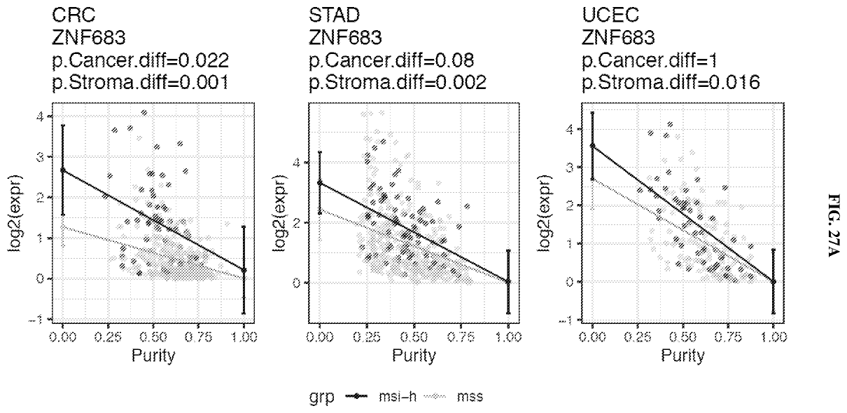

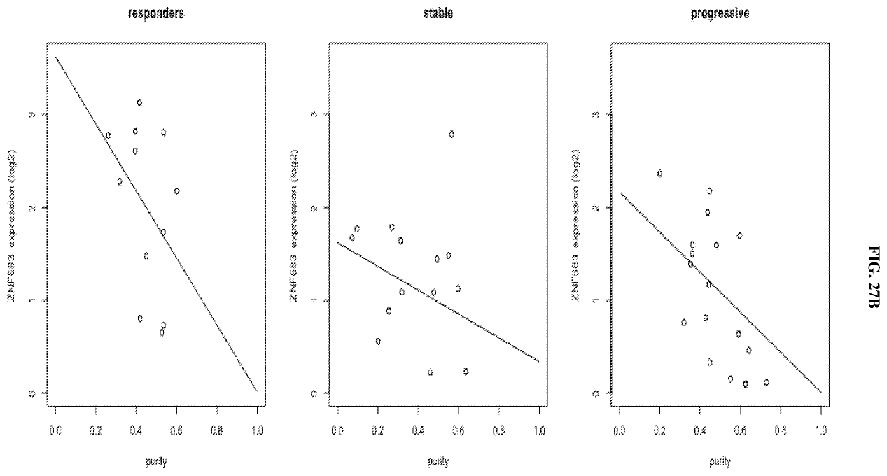

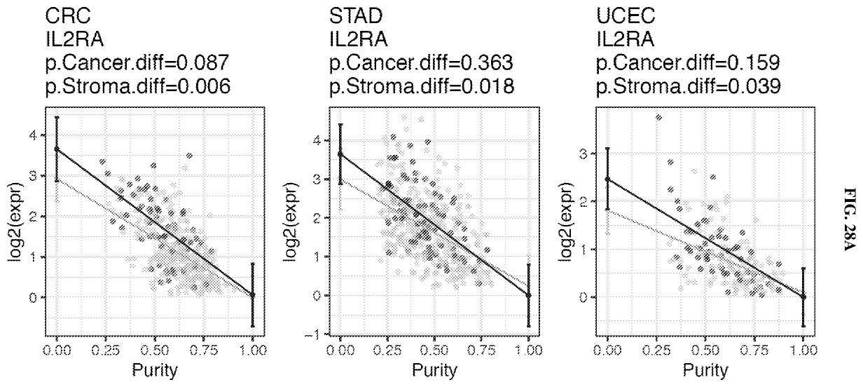

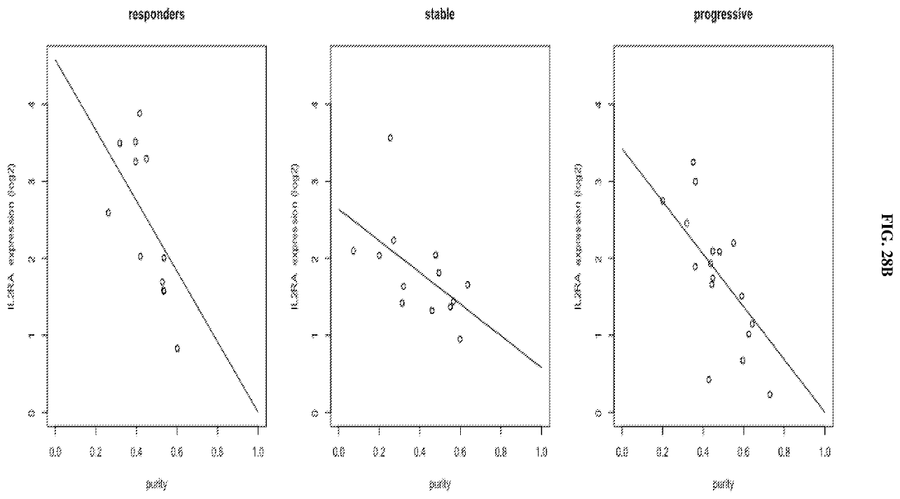

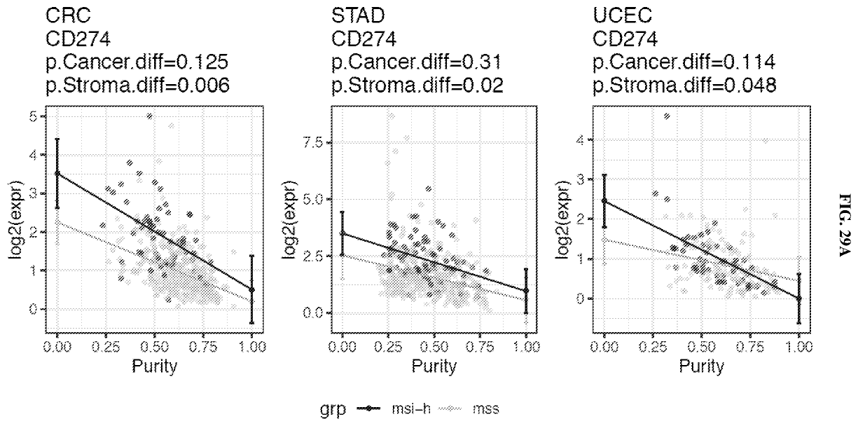

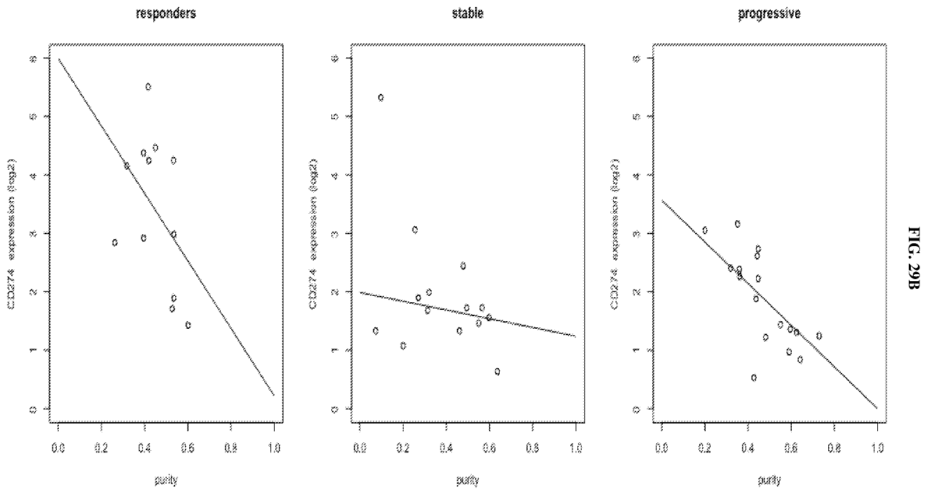

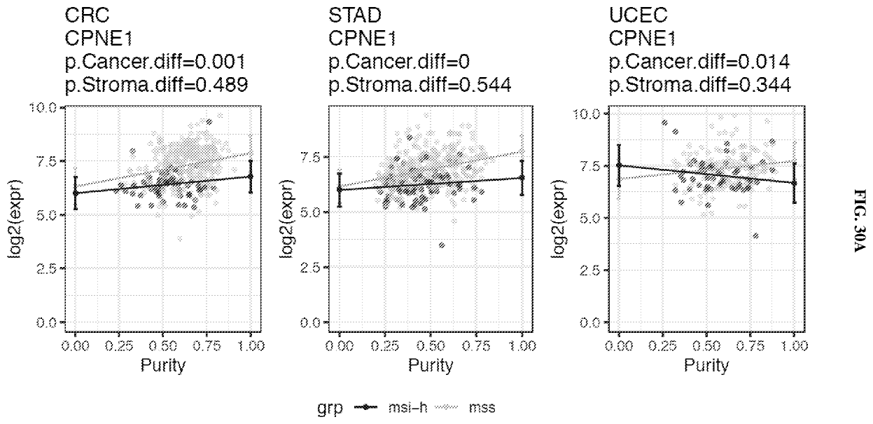

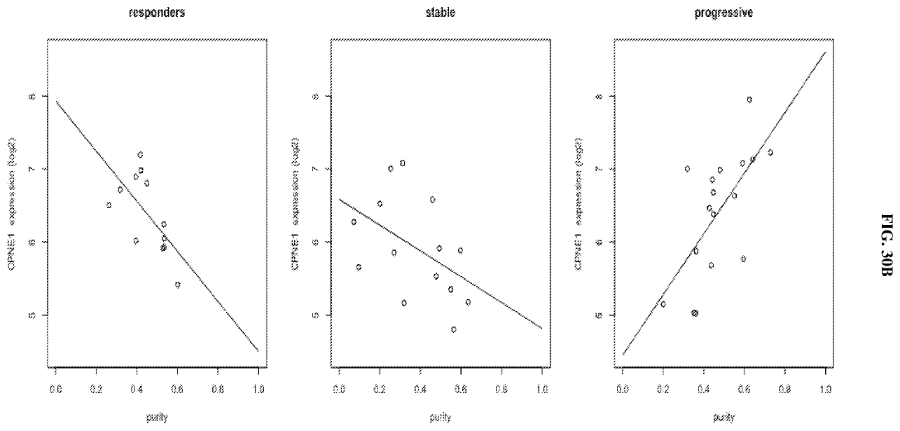

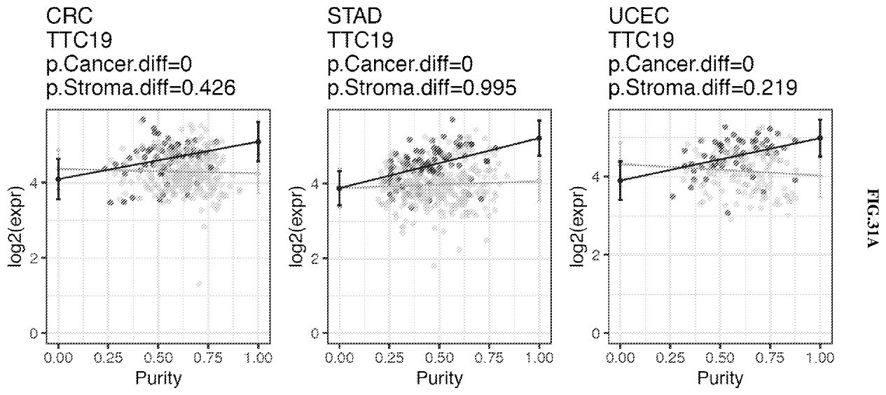

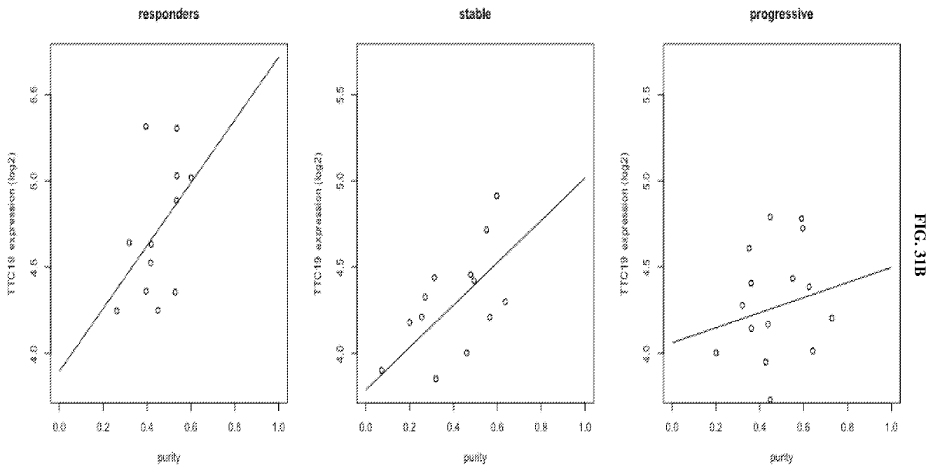

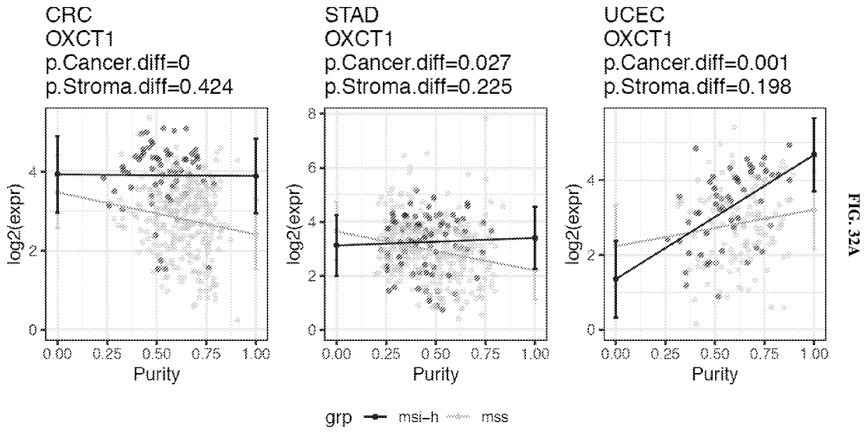

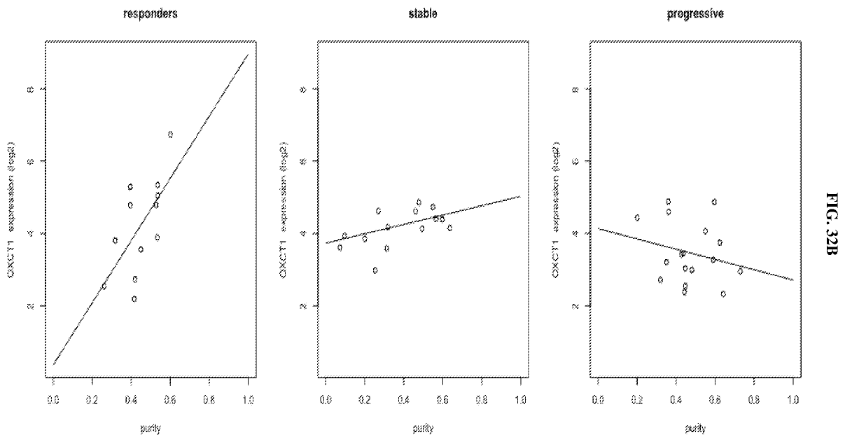

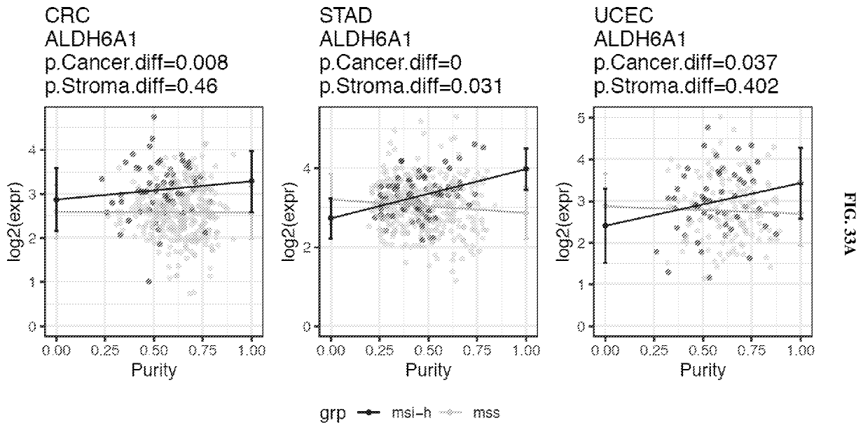

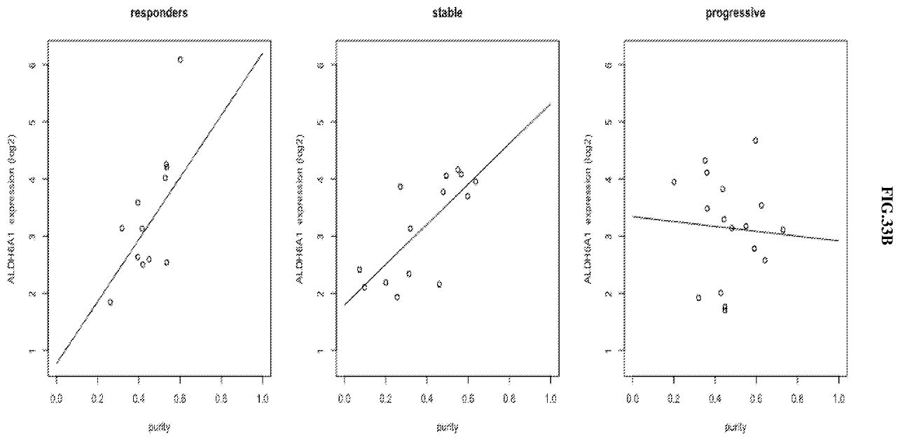

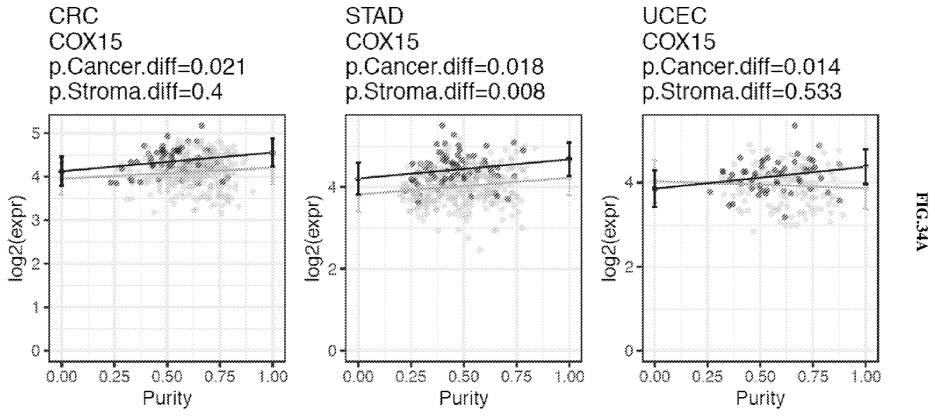

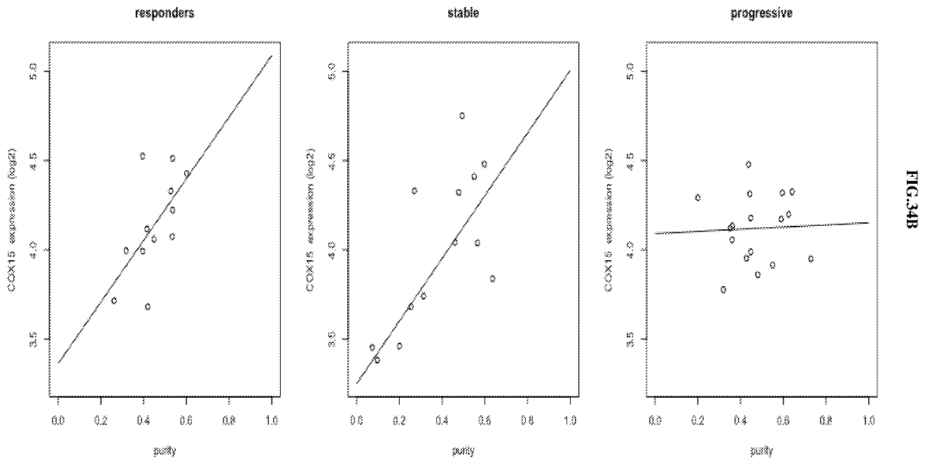

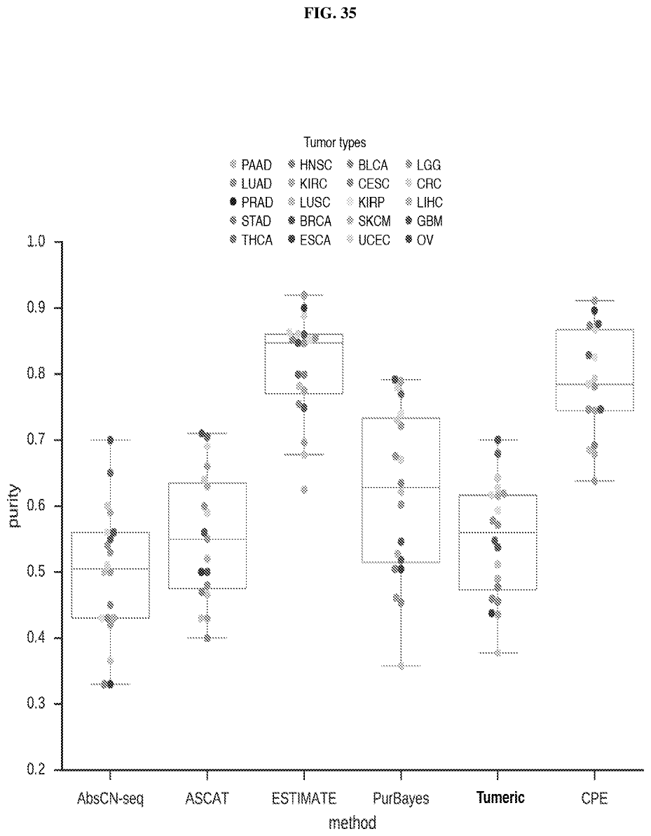

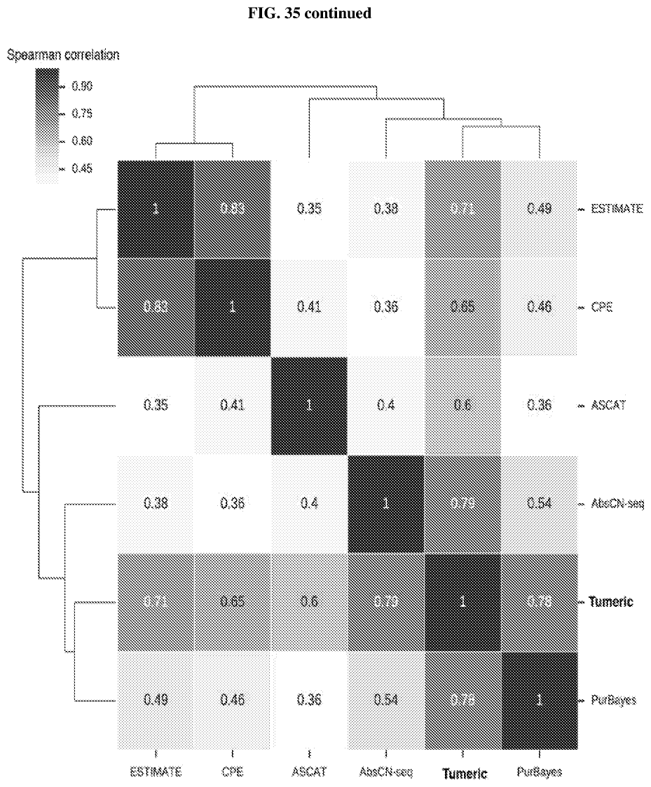

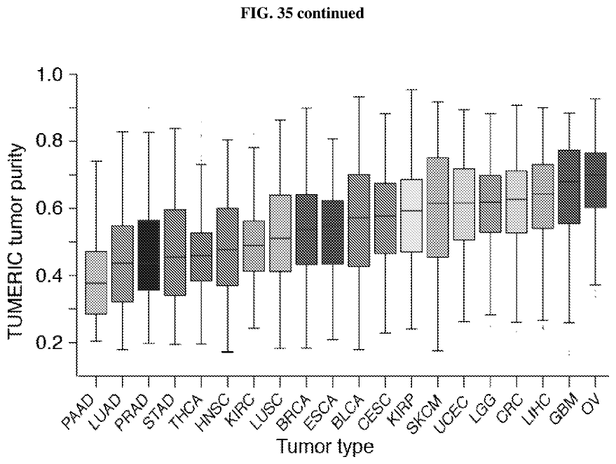

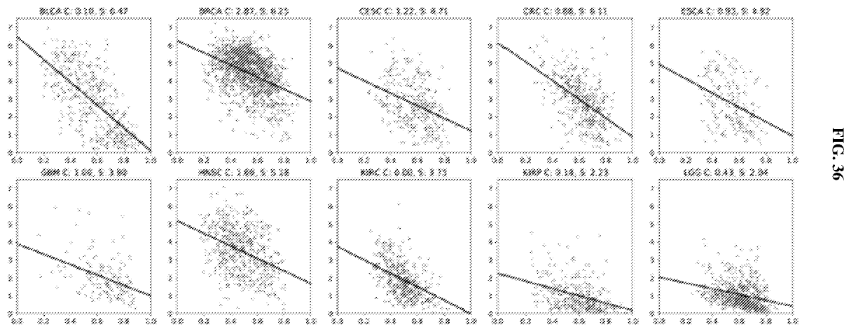

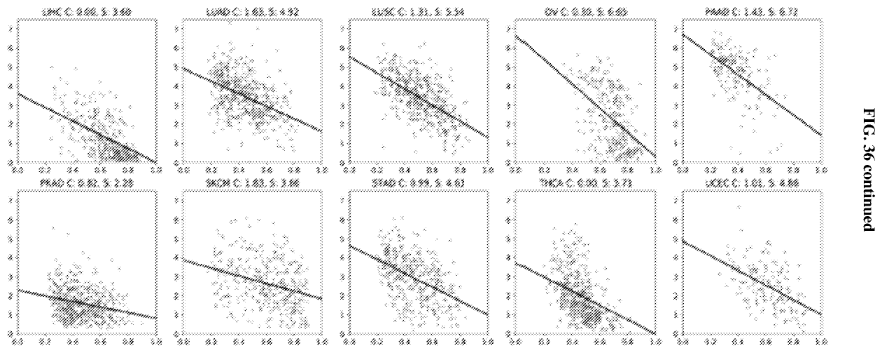

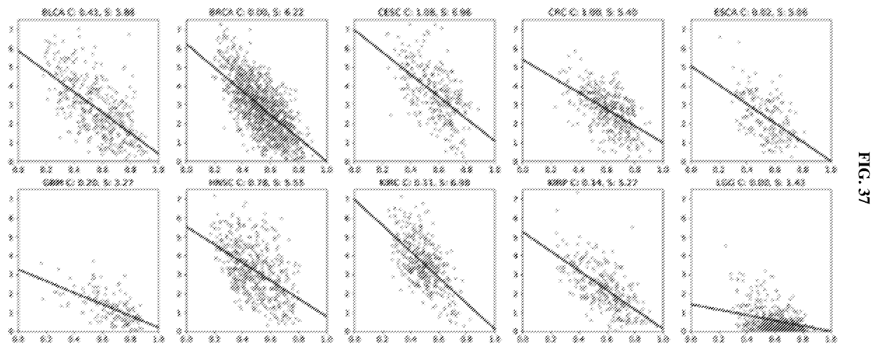

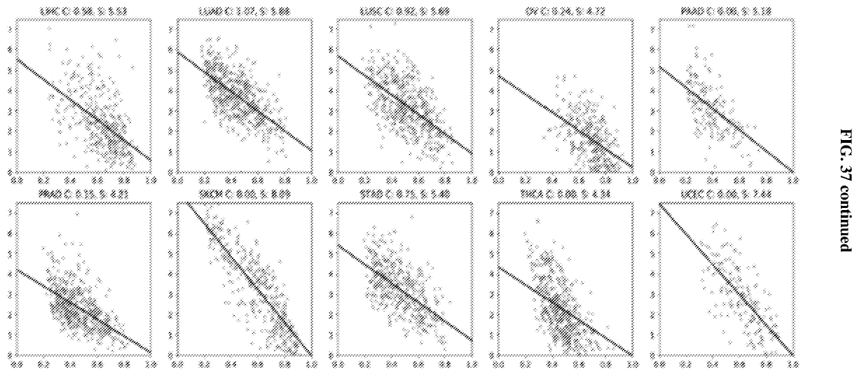

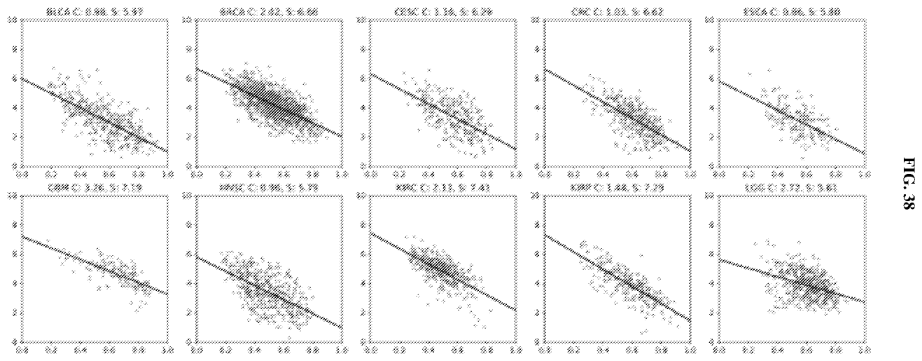

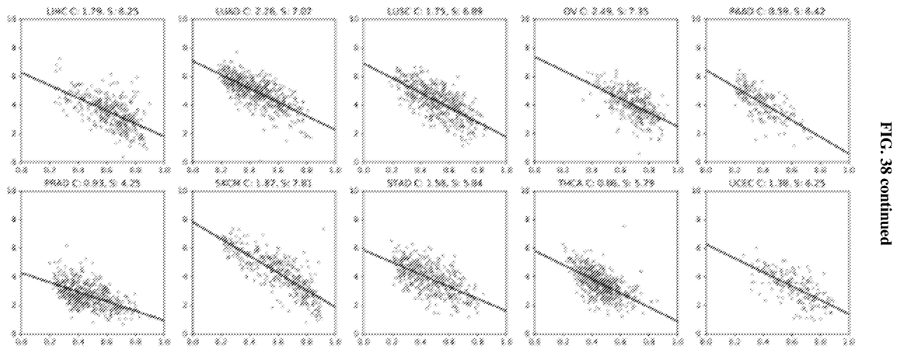

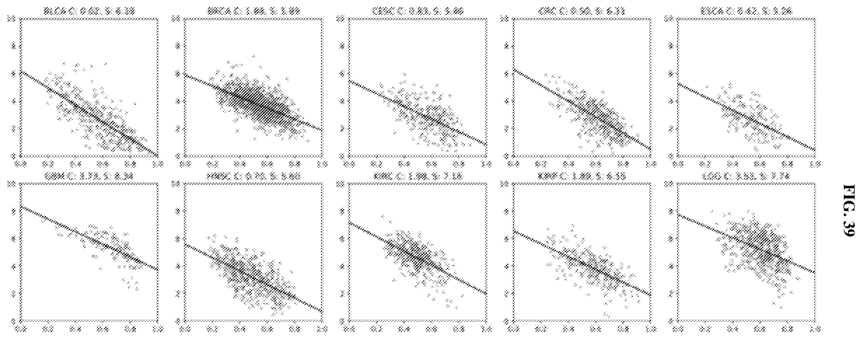

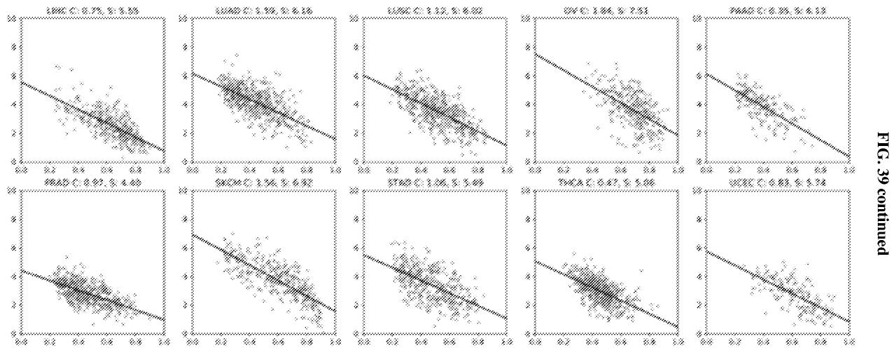

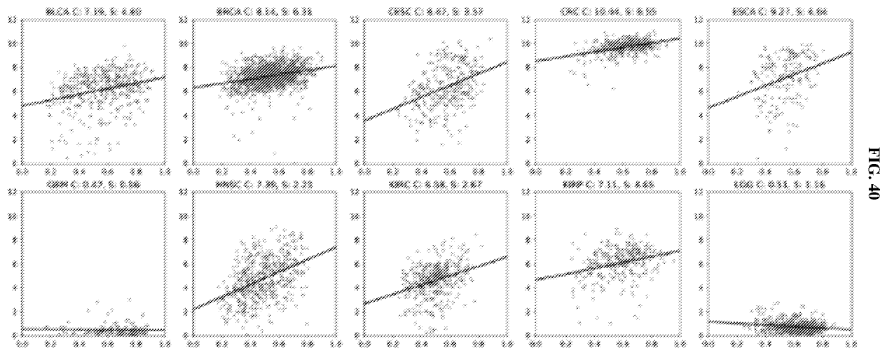

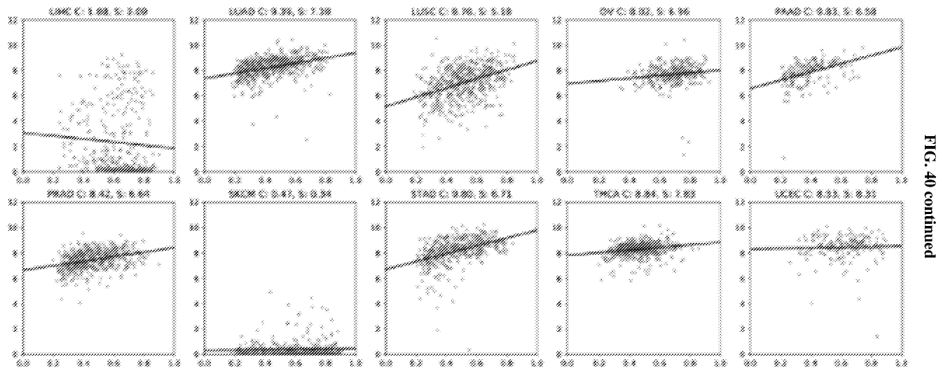

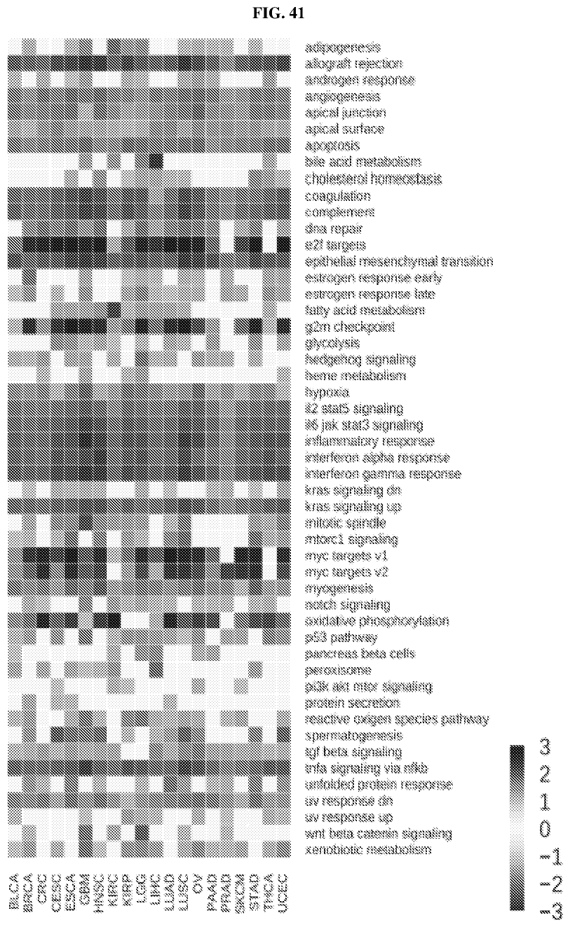

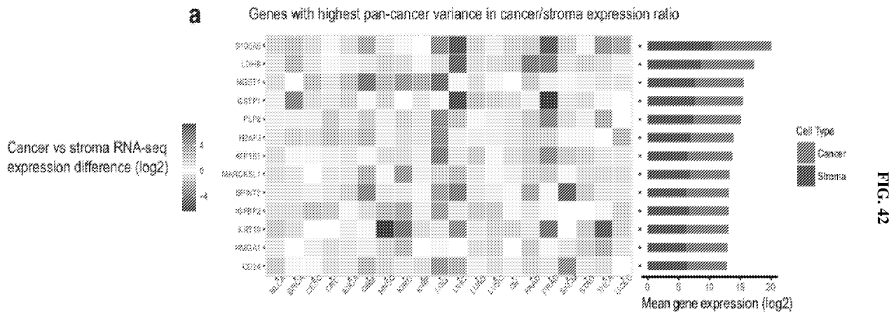

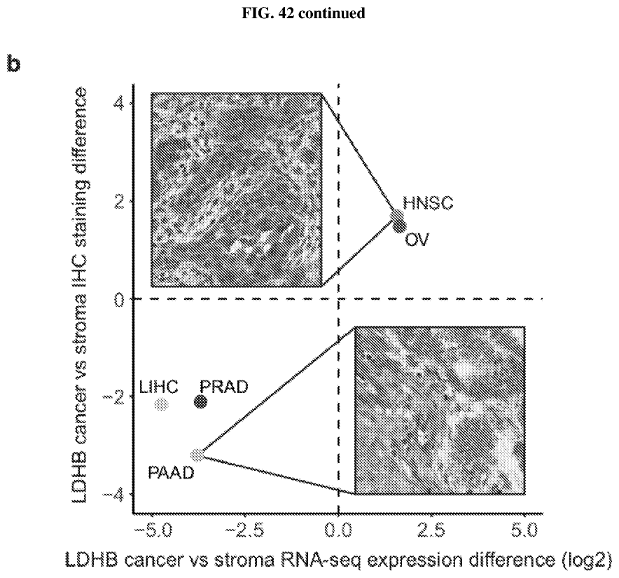

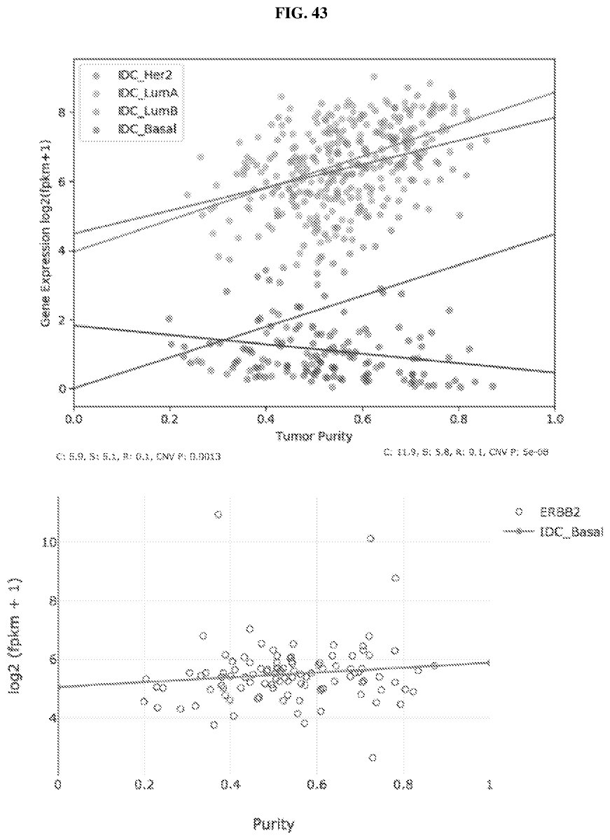

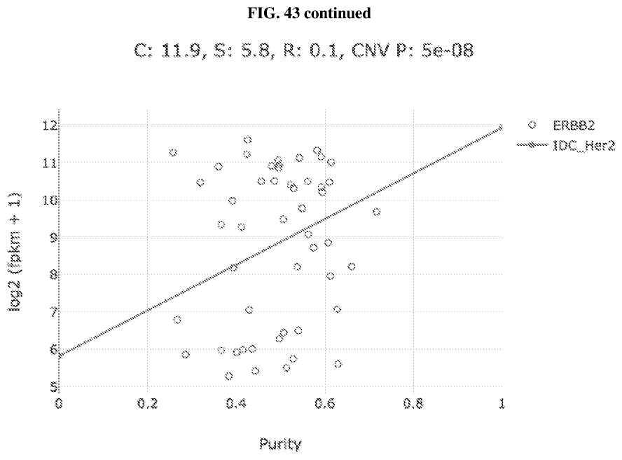

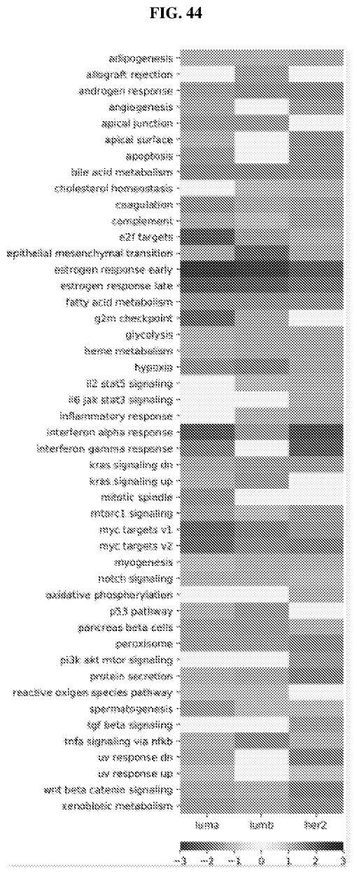

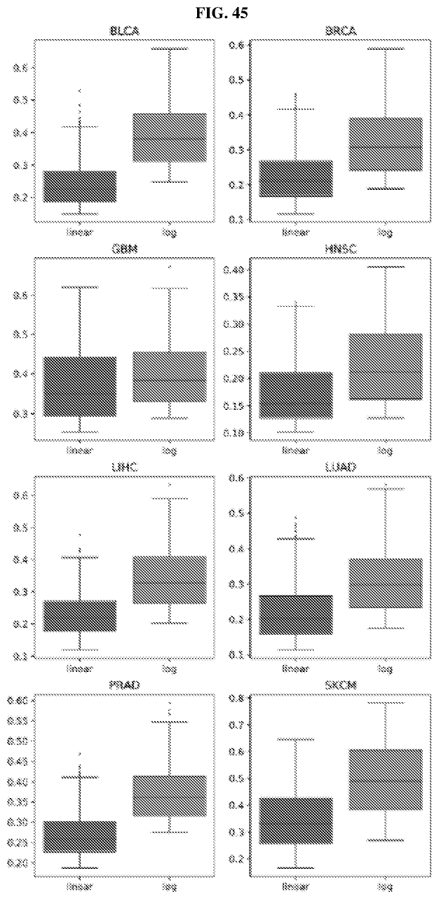

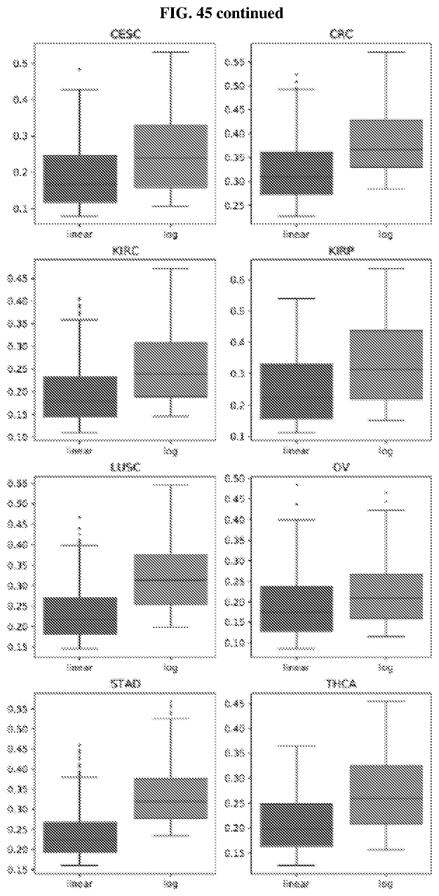

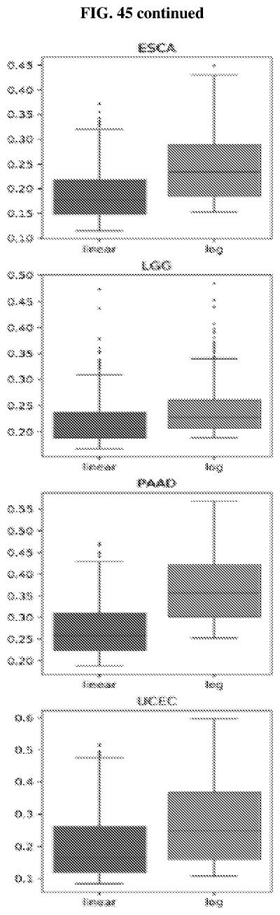

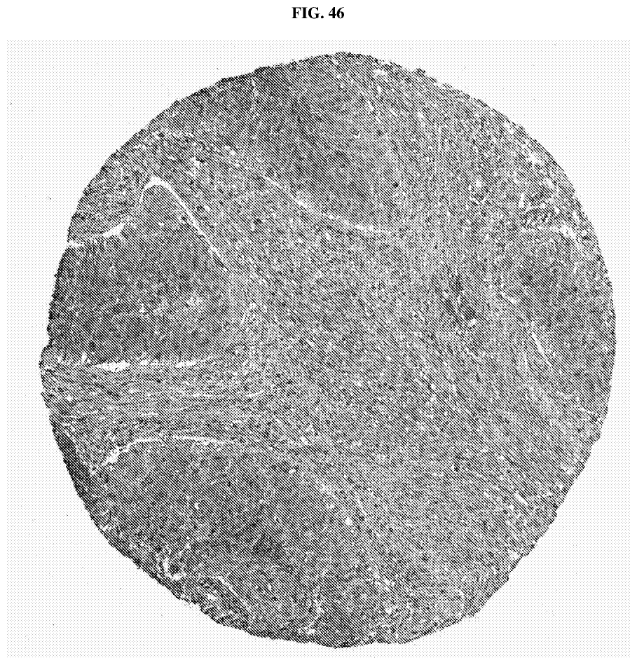

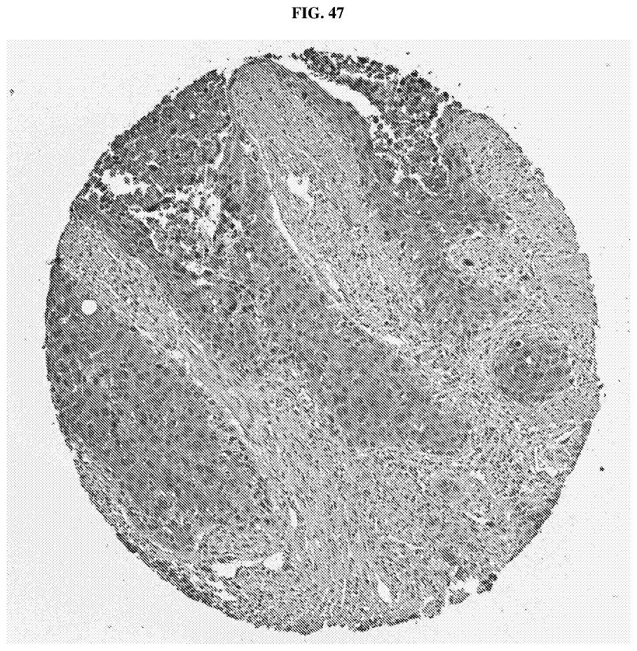

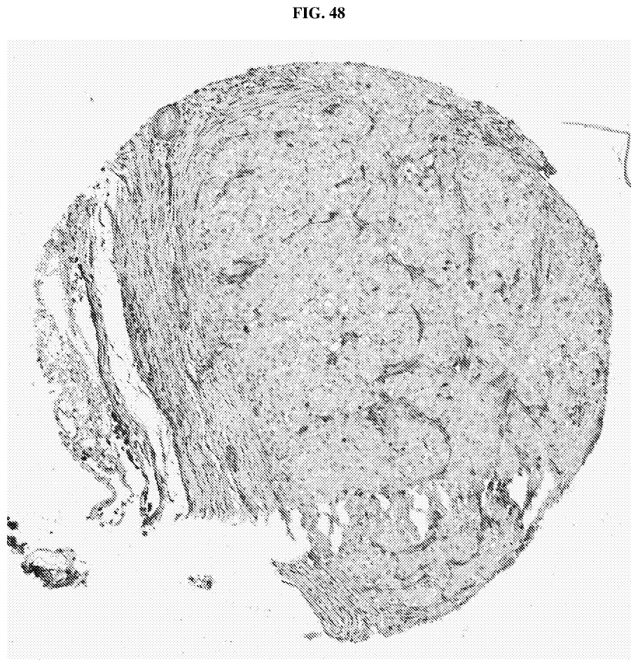

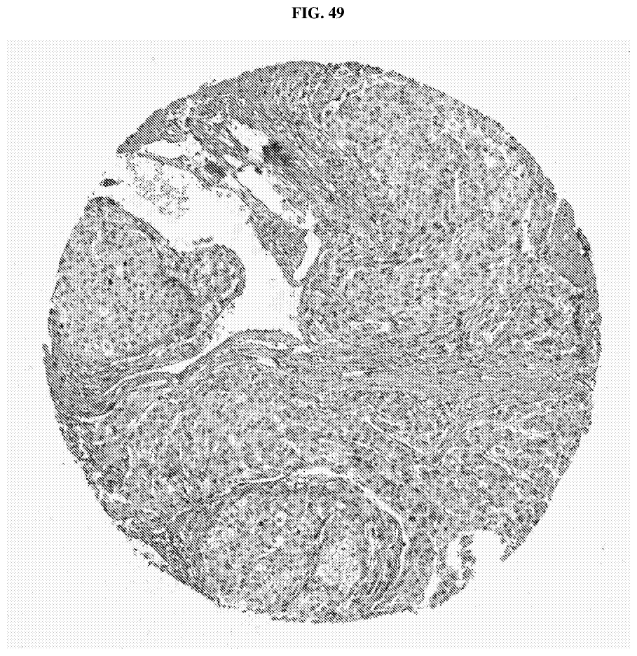

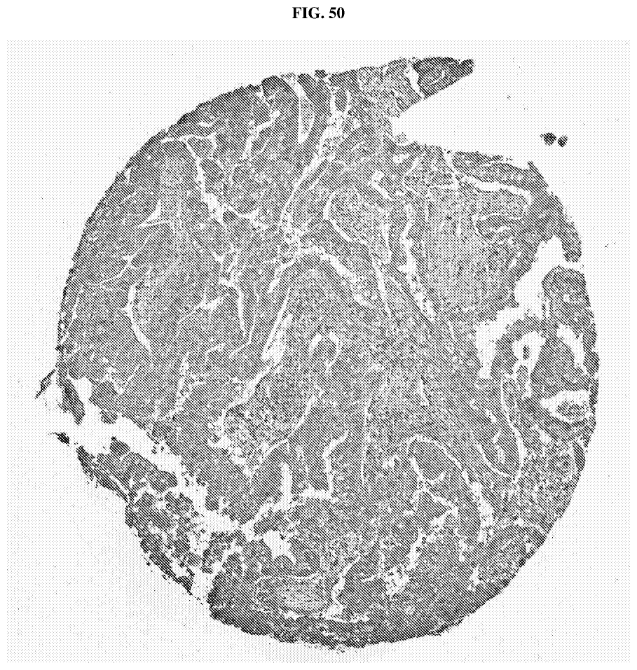

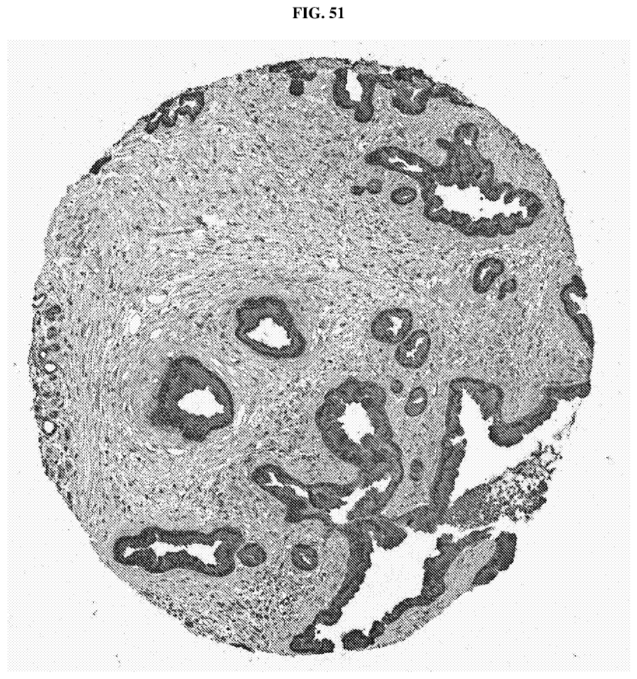

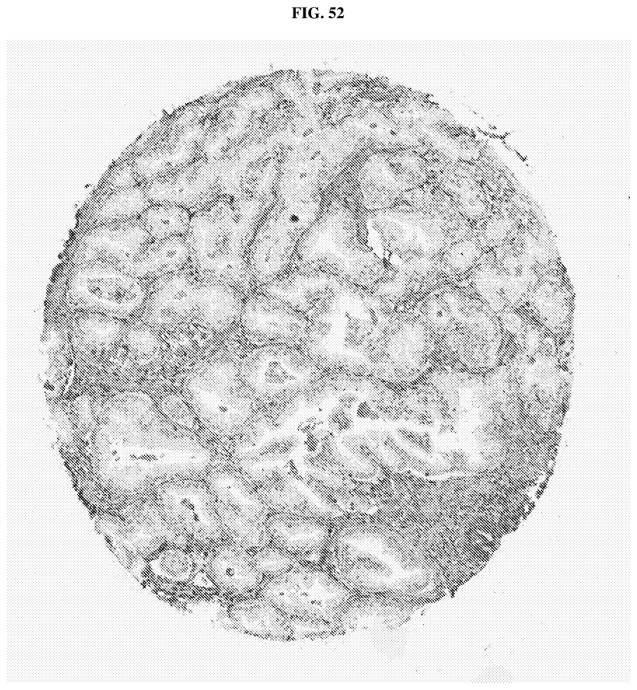

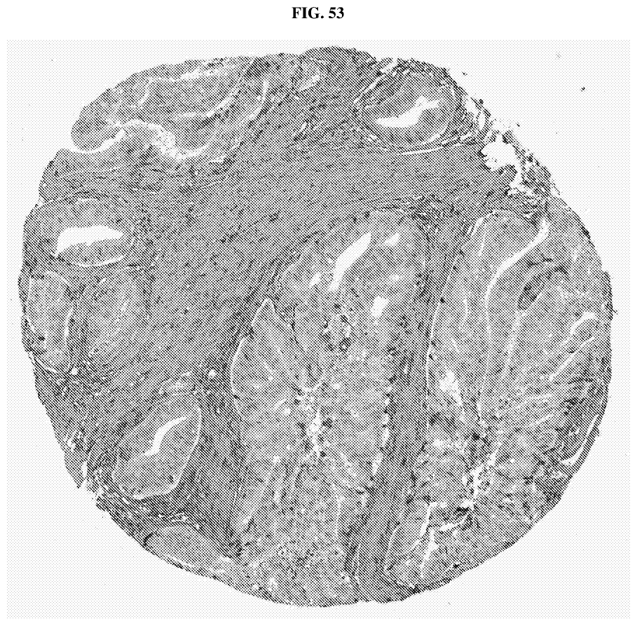

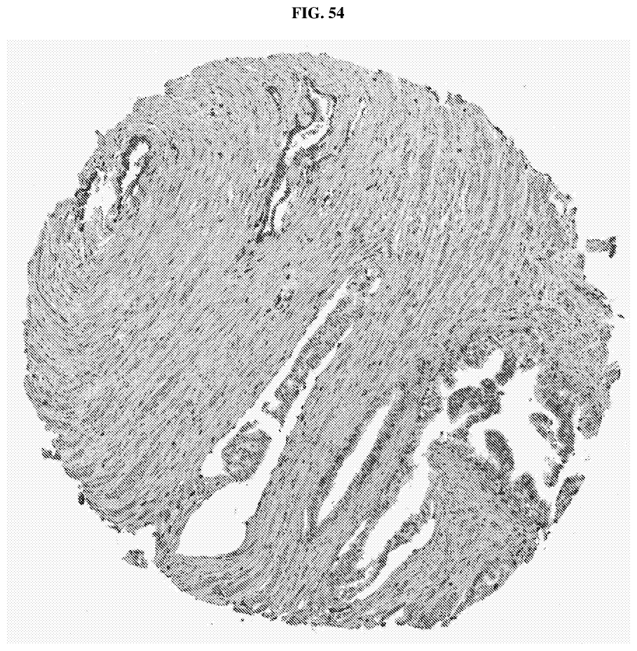

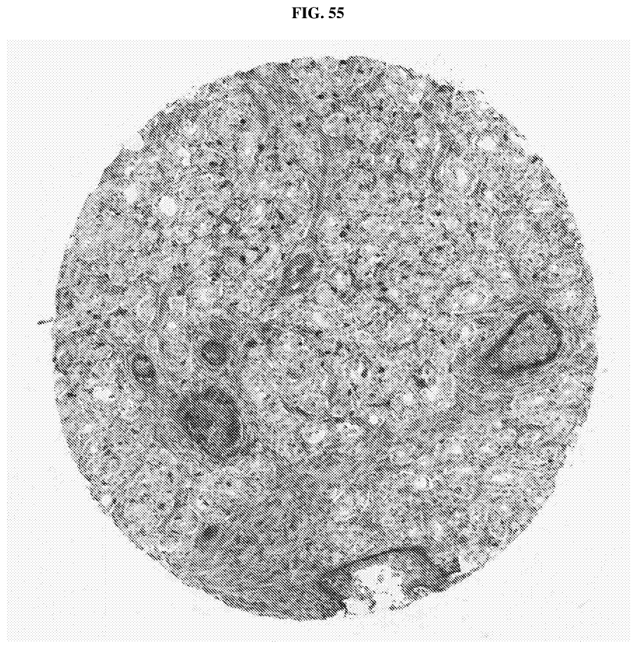

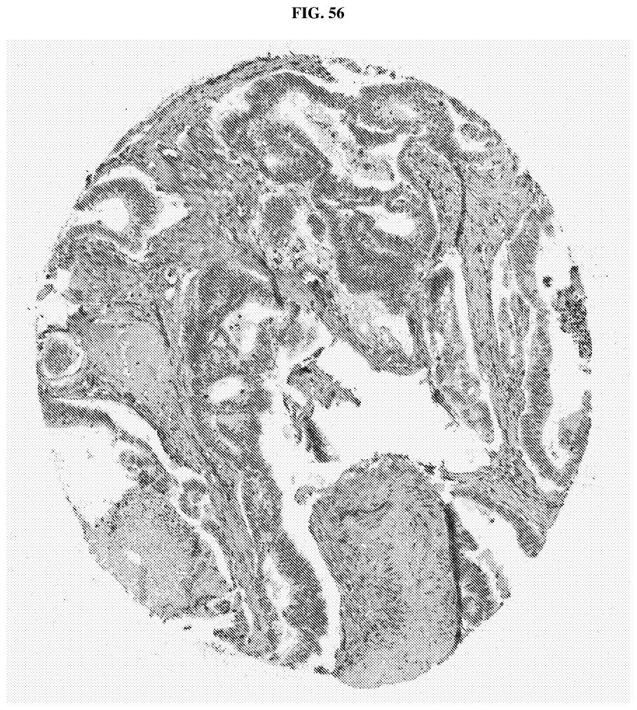

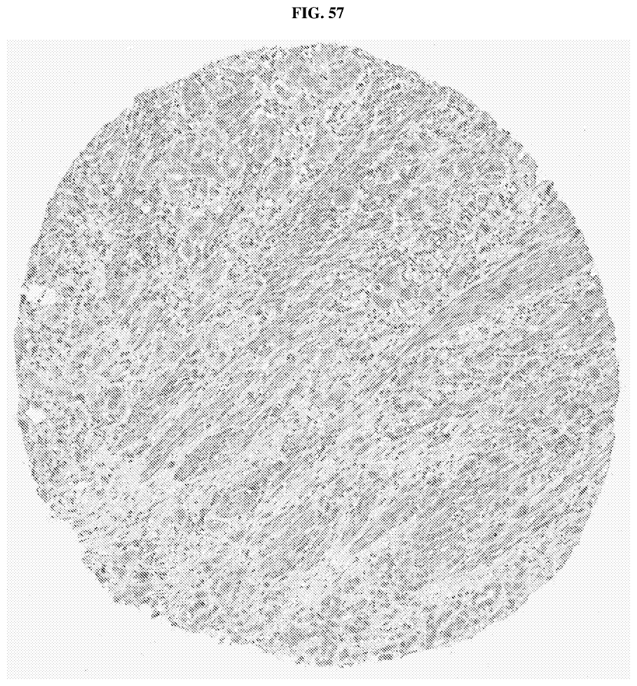

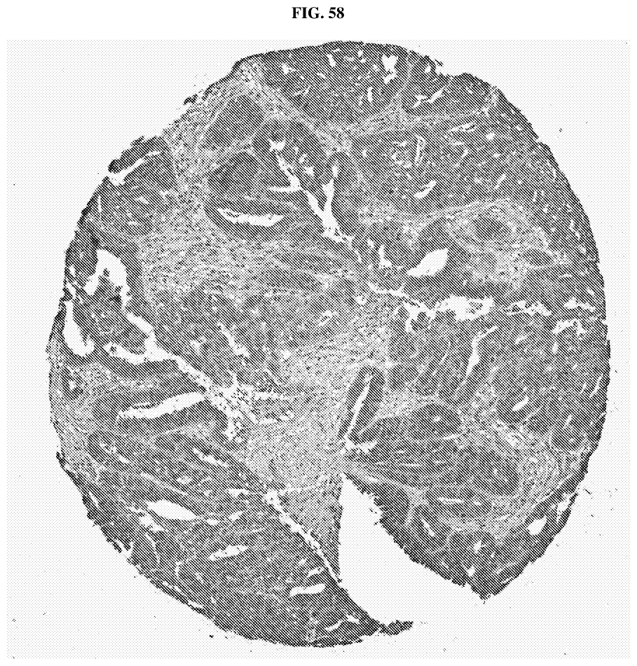

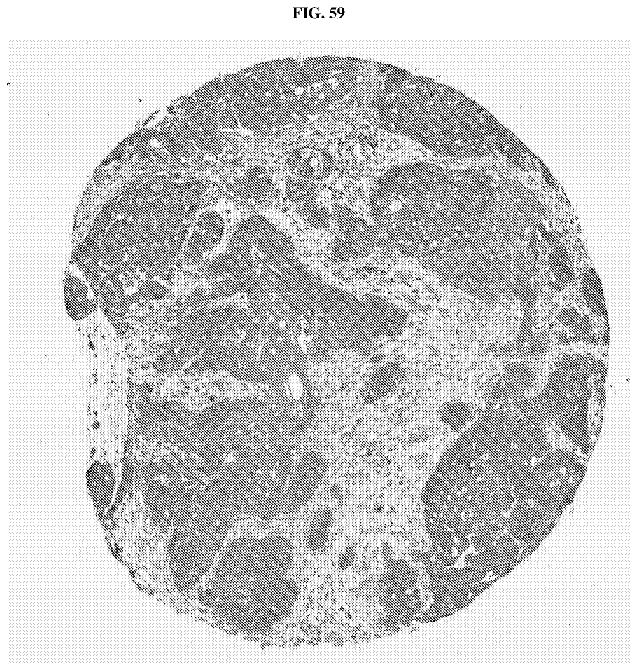

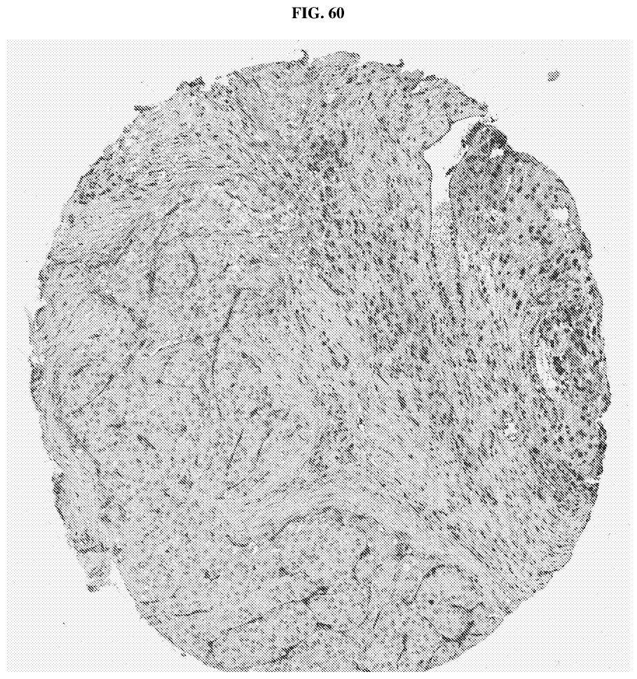

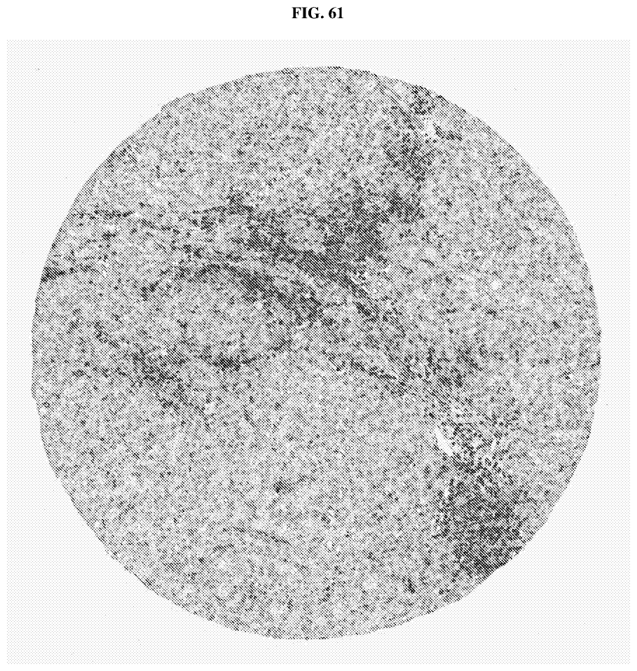

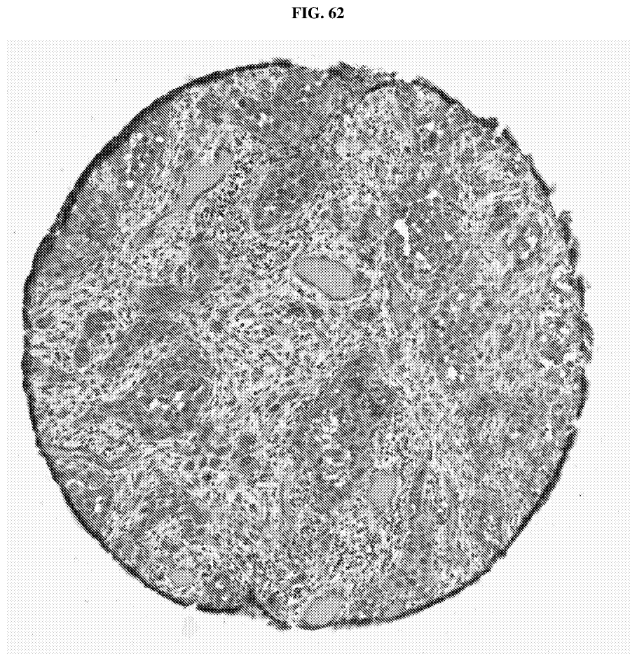

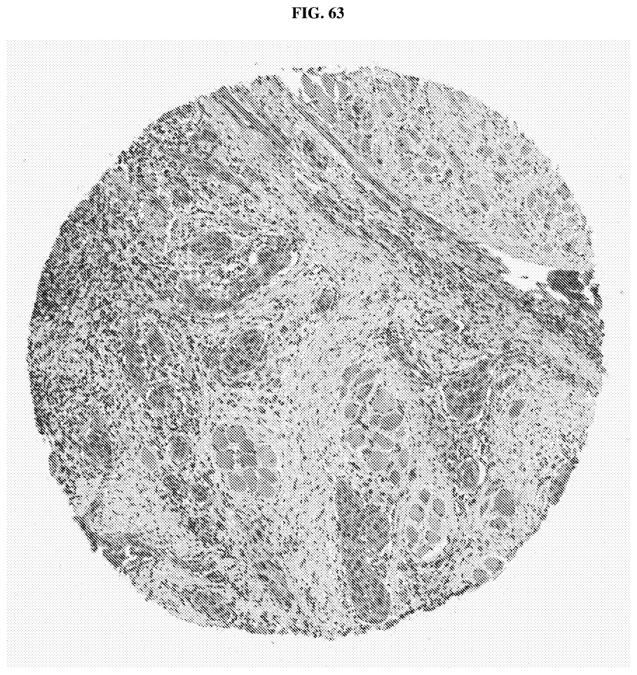

FIG. 20 shows the landscape of technologies available for high-throughput profiling of tumor transcriptomes. Existing technologies either provides high resolution (single-cell RNA-seq, sc-RNAseq) or high scalability (e.g. immuno-histochemistry IHC and bulk tumor profiling). Tumeric-Solo provides increased resolution (profiles cancer and stromal cells separately) over bulk tumor profiling, and Tumeric-Solo is easier to scale (can analyse FFPE samples) than sc-RNAseq.

FIG. 21 shows the underlying mathematical model of TUMERIC and TUMERIC-Solo. Measured bulk tumor mRNA abundance in a sample (sector/part for TUMERIC-Solo) is determined by the sum of mRNA molecules from the cancer and non-cancer-cells in that sample. Tumor purity can be estimated from DNA sequence data obtained from the same tumor sample/sector.

FIG. 22 shows a breakdown of the 8000 bulk tumours in the Cancer Genome Atlas (TCGA) that were used for the validation analysis of TUMERIC. All tumours have DNA (Exome-sequencing) and RNA (RNA-sequencing) data.

FIG. 23 shows the process by which TUMERIC and TUMERIC-Solo estimates cancer/stroma compartment proportions (tumour purity). Mutation data (DNA), Copy number number data (aCGH), and/or mRNA expression data obtained from the same tumor (sector for TUMERIC-solo) is used to produce a consensus tumor purity estimate. Purity estimates from different methods are normalized, missing data imputed, and estimates averaged for each sample/sector.

FIG. 24. IFNG: up-regulated in stroma of MSI and ICI responding tumours. FIG. 24A shows IFNG expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 24B shows data from TUMERIC applied to IFNG expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 25. FASLG: up-regulated in stroma of MSI and ICI responding tumours. FIG. 25A shows FASLG expression as a function of tumor purity in microsatellite instable (MSI, dark-grey points) and stable (MSS, light-grey points) tumors of colorectal (CRC, left), stomach (STAD, middle), and endometrial (UCEC, right) cancer. The regression lines show the TUMERIC inferred cancer and stromal cell gene expression in each cancer type and MSI/MSS subtype.

FIG. 25B shows data from TUMERIC applied to FALSG expression in patients with different responses (responders; stable disease; progressive disease) to pembrolizumab treatment. The plots show the association of measured bulk gene expression (y-axis) with estimated tumor sample purity (x-axis) for the three treatment response groups.

FIG. 26. CXCL13: up-regulated in stroma of MSI and ICI responding tumours. FIG. 26A shows CXCL13 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 26B shows data from TUMERIC applied to CXCL13 expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 27. ZNF683: up-regulated in stroma of MSI and ICI responding tumours. FIG. 27 shows ZNF683 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 27B shows data from TUMERIC applied to ZNF683 expression in patients with different responses (responders; stable disease; progressive disease) to pembrolizumab treatment. The plots show the association of measured bulk gene expression (y-axis) with estimated tumor sample purity (x-axis) for the three treatment response groups.

FIG. 28. IL2RA: up-regulated in stroma of MSI and ICI responding tumours. FIG. 28A shows IL2RA expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 28B shows data from TUMERIC applied to IL2RA expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 29. CD274/PD-L1: up-regulated in stroma of MSI and ICI responding tumours. FIG. 29A shows CD274/PD-L1 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 29B shows data from TUMERIC applied to CD274 expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 30. CPNE1: down-regulated in cancer cells of MSI and ICI responding tumours. FIG. 30A shows CPNE1 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 30B shows data from TUMERIC applied to CPNE1 expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 31. TTC19: up-regulated in cancer cells of MSI and ICI responding tumours. FIG. 31A shows TTC19 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 31B shows data from TUMERIC applied to TTC19 expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 32. OXCT1: up-regulated in cancer cells of MSI and ICI responding tumors. FIG. 32A shows OXCT1 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 32B shows data from TUMERIC applied to OXCT1 expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 33. ALDH6A1: up-regulated in cancer cells of MSI and ICI responding tumours. FIG. 33A shows ALDH6A1 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 33B shows data from TUMERIC applied to ALDH6A1 expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 34. COX15: up-regulated in cancer cells of MSI and ICI responding tumours. FIG. 34A shows COX15 expression as a function of tumour purity in microsatellite instable (MSI, dark-grey points) and stable (MSS, light-grey points) tumours of colorectal (CRC, left), stomach (STAD, middle), and endometrial (UCEC, right) cancer. The regression lines show the TUMERIC inferred cancer and stromal cell gene expression in each cancer type and MSI/MSS subtype. FIG. 34B shows data from TUMERIC applied to COX15 expression in patients with different responses (responders; stable disease; progressive disease) to pembrolizumab treatment. The plots show the association of measured bulk gene expression (y-axis) with estimated tumour sample purity (x-axis) for the three treatment response groups.

FIG. 35 first shows whisker box plots showing that tumour purity was estimated by various methods for the ˜8000 TCGA tumours across the 20 cancer types. The median purity estimated for a given method and cancer type is plotted. Tumeric is a normalized average of AbsCN-seq, ASTAC, ESTIMATE, and PurBayes (see Methods). CPE are previously published consensus purity estimates from TCGA samples and was included for comparison. To explore the concordance of different purity estimation methods, methods were clustered based on their Pearson correlation (1-r) and Ward's linkage, the data of which is provided in the second part of FIG. 35. CPE is largely based on purity estimates from ESTIMATE, and these two methods therefore expectedly cluster tightly together (r=0.83). In the third part of FIG. 35, whisker box plots show estimated tumour purity values for ˜8000 bulk tumour samples across 20 solid tumour types. Pancreatic adenocarcinoma (PAAD) tumours had very low average purity (˜39%), consistent with previous observations. The glioblastoma (GBM) and ovarian cancer (OV) samples had the highest purity estimates, likely due to tumour selection bias in the first phase of the TCGA project.

FIG. 36 Deconvolution of Fibroblast activation protein alpha (FAP) gene expression across different cancer types. Inferred cancer (C) and stromal (S) cell gene expression (log 2 FPKM+1) listed for each cancer type.

FIG. 37 Deconvolution of T-cell surface glycoprotein CD3 delta chain (CD3D) gene expression across different cancer types. Inferred cancer (C) and stromal (S) cell gene expression (log 2 FPKM+1) listed for each cancer type.

FIG. 38 Deconvolution of CD4 gene expression across different cancer types. Inferred cancer (C) and stromal (S) cell gene expression (log 2 FPKM+1) listed for each cancer type.

FIG. 39 Deconvolution of Colony stimulating factor 1 receptor (CSF1R) gene expression across different cancer types. Inferred cancer (C) and stromal (S) cell gene expression (log 2 FPKM+1) listed for each cancer type.

FIG. 40 Deconvolution of Epithelial cell adhesion molecule (EPCAM) gene expression across different cancer types. Inferred cancer (C) and stromal (S) cell gene expression (log 2 FPKM+1) listed for each cancer type.

FIG. 41 shows a heat map of normalized enrichment scores (NES) of MSigDB Hallmark Gene Sets obtained by GSEA pre-ranked analysis of log 2((Cancer_FPKM+1)/(Stroma_FPKM+1)) following deconvolution. Immune system related pathways like inflammatory response, interferon alpha/gamma response etc. are upregulated in stroma whereas known cancer-cell specific pathways like MYC targets, G2M checkpoint, DNA repair are upregulated. Cells with red/blue colors have FDR<=0.25, cells in white have FDR>0.25.

FIG. 42 a) Genes with highly variable cancer vs. stroma mRNA expression differences across cancer types were identified. b) Immunohistochemistry (IHC) staining data was compared to RNA-seq data for the gene with highest (S100A6) and second-highest (LDHB) abundance.

FIG. 43. Deconvolution of gene expression for estrogen receptor 1 (ESR1) in breast cancer subtypes of Invasive Ductal Carcinoma (IDC) Luminal A (IDC_LumA), Luminal B (IDC_LumB), Basal (IDC_Basal) and HER2 (IDC_Her2) (first graph). As expected, ESR1-negative HER2 and Basal subtypes have low expression of ESR1. Similarly, ESR1-positive subtypes LumA and LumB have very high expression of ESR1 in cancer cells (fpkm˜387 in case of LumA and fpkm˜221 in case of LumB). Deconvolution of ERBB2/HER2 expression in Basal (left) and HER2+(right) subtypes (second and third graph). Deconvolution of ERBB2 expression in HER2 tumours also took into account frequent HER2 amplification events (see methods).

FIG. 44. Gene set enrichment analysis (GSEA), comparing cancer compartment gene expression (that is, cancer cell gene expression) in Luminal A (luma), Luminal B (lumb), and HER2 (her2) tumors with Basal tumors. The heatmap shows GSEA normalized enrichment scores (NES) for individual gene sets for the three different subtype comparisons, blue colours (negative NES) reflect gene sets upregulated in Basal relative to other cancer types. Cells with light/dark grey colours have FDR<=0.25, while cells in white have FDR>0.25.

FIG. 45 Comparison of deconvolution using linear and log-transformed RNA-seq gene expression (FPKM+1). Plots shows top 5% of purity vs. gene expression coefficient of determination (R2) obtained for each transformation. Across all cancer types, tumor purity has overall stronger linear correlation with log transformed RNA-seq gene expression data.

FIG. 46 shows an IHC image of a BLCA (Bladder Urothelial Carcinoma) tumour sample stained with S100A6.

FIG. 47 shows an IHC image of a BLCA (Bladder Urothelial Carcinoma) tumour sample stained with S100A6.

FIG. 48 shows an IHC image of a LIHC (Liver Hepatocellular Carcinoma) tumour sample stained with S100A6.

FIG. 49 shows an IHC image of a LIHC (Liver Hepatocellular Carcinoma) tumour sample stained with S100A6.

FIG. 50 shows an IHC image of a PAAD (Pancreatic Adenocarcinoma) tumour sample stained with S100A6.

FIG. 51 shows an IHC image of a PAAD (Pancreatic Adenocarcinoma) tumour sample stained with S100A6.

FIG. 52 shows an IHC image of a PRAD (Prostate Adenocarcinoma) tumour sample stained with S100A6.

FIG. 53 shows an IHC image of a PRAD (Prostate Adenocarcinoma) tumour sample stained with S100A6.

FIG. 54 shows an IHC image of a PRAD (Prostate Adenocarcinoma) tumour sample stained with LDHB.

FIG. 55 shows an IHC image of a PRAD (Prostate Adenocarcinoma) tumour sample stained with LDHB.

FIG. 56 shows an IHC image of a PAAD (Pancreatic Adenocarcinoma) tumour sample stained with LDHB.

FIG. 57 shows an IHC image of a PAAD (Pancreatic Adenocarcinoma) tumour sample stained with LDHB.

FIG. 58 shows an IHC image of an OV (Ovarian Serous Cystadenocarcinoma) tumour sample stained with LDHB.

FIG. 59 shows an IHC image of an OV (Ovarian Serous Cystadenocarcinoma) tumour sample stained with LDHB.

FIG. 60 shows an IHC image of a LIHC (Liver Hepatocellular Carcinoma) tumour sample stained with LDHB.

FIG. 61 shows an IHC image of a LIHC (Liver Hepatocellular Carcinoma) tumour sample stained with LDHB.

FIG. 62 shows an IHC image of a HNSC (Head and Neck Squamous Cell Carcinoma) tumour sample stained with LDHB.

FIG. 63 shows an IHC image of a HNSC (Head and Neck Squamous Cell Carcinoma) tumour sample stained with LDHB.

### DEFINITIONS

As used herein, the term ‘tumour type’ refers to: a tumour selected by its anatomy, such as breast cancer or lung cancer; a tumour selected by cancer type, such as carcinoma or melanoma; tumour subtypes of the same cancer type; or tumours that are treated with the same treatment type. Examples of such treatments are, but are not limited to gefitinib, erlotinib and afatinib for the treatment of cancer related to EGFR; OSI-906 (linsitinib) for the treatment of cancer related to IGF1R; everolimus (also known as RAD001) and sirolimus for the treatment of cancer related to mTOR; BKM120 (buparlisib) and BYL719 (alpelisib) for the treatment of cancer related to PIK3CB and PIK3R3; idelalisib for the treatment of cancer related to PIK3CD and dacomatinib and lapatinib for the treatment of cancer related to ERBB4, or combinations thereof. In one example, the anti-cancer drug used for treating EGFR-related cancers is, but is not limited to, gefitinib, erlotinib, afatinib or combinations thereof. In another example, the anti-cancer drug used for treating mTOR-related cancers is, but is not limited to, everolimus (RAD001), sirolimus, or combinations thereof. In another example, the anti-cancer drug used for treating IGF1R-related cancers is, but is not limited to, linsitinib. In another example, the anti-cancer drug used for treating PIK3CB and PIK3R3-related cancers is, but is not limited to, BKM120 (buparlisib), BYL719 (alpelisib) or combinations thereof. In another example, the anti-cancer drug used for treating PIK3CD-related cancers is, but is not limited to, idelalisib. In another example, the anti-cancer drug used for treating ERBB4-related cancers is, but is not limited to, dacomatinib, lapatinib, or combinations thereof. In one example, the anti-cancer drug is a tyrosine kinase inhibitor. In another example, the tyrosine kinase inhibitor is an EGFR inhibitor. In yet another example, the tyrosine kinase inhibitor is, but is not limited to, gefitinib, erlotinib, erlotinib HCl, lapatinib, dacomitinib, TAE684, afatinib, dasatinib, saracatinib, veratinib, AEE788, WZ4002, icotinib, osimertinib, B11482694, ASP8273, EGF816, AZD3759, cetuximab, necitumumab, pannitumumab, nimotuzumab and combinations thereof. In a further example, the tyrosine kinase inhibitor is, but is not limited to, gefitinib, erlotinib, lapatinib and combinations thereof. In one example, the tumour type can be, but is not limited to, BLCA, BRCA, CESC, CRC (COAD and READ combined), ESCA, GBM, HNSC, KIRC, KIRP, LGG, LIHC, LUAD, LUSC, OV, PAAD, PRAD, SKCM, STAD, THCA and UCEC, as referenced in the TCGA database.

As used herein, the term “scoring” refers to the process of ranking genes, biomarkers or therapeutic targets. The term “scoring” when used in the present application can also be used synonymously with the term “ranking”. For example, in a cohort of cancer patients (TUMERIC) or an individual cancer patient (TUMERIC-solo), all genes can be scored or ranked by their inferred expression in cancer cells to identify top-ranked candidate therapeutic targets.

As used herein, the term “tumour purity value” refers to an estimated fraction of cancerous cells out of all cells present in the tumour. In the context of the present disclosure, the term “cancer cells” and “malignant cells” are used interchangeably. The tumour purity value of a given tumour can, for example, be estimated from somatic mutation variant allele frequencies (VAFs) measured in a given sample. For example, if a known (clonal) cancer driver mutation is measured with a variant allele frequency (VAF) of 0.2 (20%) in gene X, and gene X is not altered by somatic copy number alterations in the given sample (gene X has 2 alleles/chromosomes in the cancer cells), this variant allele frequency (VAF) can be explained by a tumour comprising 40% cancer cells (1 mutated allele and 1 wildtype allele) and 60% non-cancer (2 wildtype alleles). Since many genes are mutated in tumours, the purity value is then given by the consensus value that best fits all the observed variant allele frequencies (VAFs).

As used herein, the term “variant allele frequency (VAF)” refers to the relative frequency of an allele (variant of a gene) at a particular locus in a population, expressed as a fraction or percentage of the entire population. In other words, the variant allele frequency (VAF) represents the fraction of all chromosomes in the population that carry that specific allele.

As used herein, the term “robust” and the term “accurate can be used interchangeably.

As used herein, the term “TANTIGEN” refers to the tumour T cell antigen database developed and maintained by Bioinformatics Core at Cancer Vaccine Center, Dana-Farber Cancer Institute, and as referred to in Cancer Immunol Immunother. 2017 June; 66(6):731-735. (doi: 10.1007/s00262-017-1978-y. Epub 2017 Mar. 9). The Tumour T cell antigen database is a data source and analysis platform for cancer vaccine target discovery focusing on human tumour antigens that contain HLA ligands and T cell epitopes. It catalogues more than 1000 tumour peptides from 292 different proteins. The database also provides information on T cell epitopes and HLA ligands with full references, gene expression profiles, antigen isoforms, and mutations. Predicted binding peptides of 15 HLA Class I and Class II alleles are also included in the database.

As used herein, the term “Gene Ontology” refers to the Gene Ontology Resource database which is a source of information on the functions of genes, and is maintained by Open Biological Ontologies Foundry.

As used herein, the term “TCGA” refers to The Cancer Genome Atlas Program run and maintained by the National Cancer Institute (BG 9609 MSC 9760, 9609 Medical Center Drive, Bethesda, Md. 20892-9760, USA.)

As used herein, the term “Human Protein Atlas” refers to a Swedish-based program initiated in 2003 with the aim to map all the human proteins in cells, tissues and organs using integration of various omics technologies, including antibody-based imaging, mass spectrometry-based proteomics, transcriptomics and systems biology. All the data in the knowledge resource is available online and is open access to allow scientists both in academia and industry to freely access the data for exploration of the human proteome. The Human Protein Atlas consists of six separate parts, each focusing on a particular aspect of the genome-wide analysis of the human proteins; the Tissue Atlas showing the distribution of the proteins across all major tissues and organs in the human body, the Cell Atlas showing the subcellular localization of proteins in single cells, the Pathology Atlas showing the impact of protein levels for survival of patients with cancer, the Blood Atlas, the Brain Atlas and the Metabolic Atlas.

As used herein, the term “cBioPortal” refers to an online portal for cancer genomics. The cBioPortal for Cancer Genomics was originally developed at Memorial Sloan Kettering Cancer Center. The public cBioPortal site is hosted by the Center for Molecular Oncology at the Memorial Sloan Kettering Cancer Center. The cBioPortal software is now available under an open source license via GitHub. The software is now developed and maintained by a multi-institutional team, consisting of the Memorial Sloan Kettering Cancer Center, the Dana Farber Cancer Institute, Princess Margaret Cancer Centre in Toronto, Children's Hospital of Philadelphia, The Hyve in the Netherlands, and Bilkent University in Ankara, Turkey.

As used herein, the term “Genomic Data Commons” refers to is a research program of the National Cancer Institute (NCI; NCI Center for Cancer Genomics (CCG), 31 Center Drive, Bldg. 31, Suite 3A20, Bethesda, Md. 20892).

As used herein, the term “cancer compartment” refers to cancer cells. For example, as used herein, Tumeric-solo is used to estimate/infer the expression of genes in the cancer cells/compartment. Genes are rank/ordered from high to low based on this inferred cancer expression level.

The embodiments illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by present embodiments and optional features, modification and variation of the embodiments embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

### DETAILED DESCRIPTION OF THE PRESENT INVENTION

Described herein is an approach to quantify, genome-wide and high-throughput, molecular activity (such as mRNA, DNA methylation, or protein expression) in cancer and non-cancer cells of individual patient tumours, which has specific applications for discovering new biomarkers and treating individual patients based on aberrant molecular activities. Signalling between cancer and non-malignant (for example, stromal) cells in the tumour microenvironment is difficult to study within patient tumours. Thus, disclosed herein is a data-driven method for deconvolution of cancer and stromal cell transcriptomes and inference of cell-cell signalling crosstalk in bulk tumour tissue. With this approach, crosstalk common across different solid tumour types and inferred modes of EGF-family crosstalk in subtypes of breast cancer are advantageously identified in bulk tumour tissue. The method is further demonstrated to be advantageous in nomination of novel drug targets, nomination of treatments in a patient-specific manner, as well as identification and quantification of biomarkers of immune checkpoint inhibition anti-cancer therapy.

In accordance with a present embodiment, there is disclosed a combined experimental-computational method/algorithm (hereinafter also referred to as “TUMERIC-solo”) for inferring cancer and non-cancer molecular activity in an individual bulk tumour sample. The combined experimental-computational method/algorithm in accordance with the present embodiment can be applied to any type of molecular data (for example, mRNA expression (RNA-sequencing), mRNA transcript isoform expression, protein expression (using iTRAQ), or epigenetic profiling) co-extracted from, for example, different physical sections/sectors of a bulk tumour sample. The combined experimental-computational method/algorithm in accordance with the present embodiment requires as input both DNA and molecular data from N sectors of a single bulk tumour sample, and outputs estimates of molecular activity/expression in the cancer and non-cancer cells of that tumour sample. The data disclosed herein below validates the use of the combined experimental-computational method/algorithm in accordance with the present embodiment for RNA-sequencing and protein using a cohort of bulk tumour samples from different patients.

The combined experimental-computational method/algorithm in accordance with the present embodiment also encompasses a method for treating a patient tumour based on specific molecular signals in cancer or non-cancer cells of an individual tumour. For example, a sample of the patient's tumour could be analysed with TUMERIC-solo, and the patient could be treated according to the measured molecular activities in the cancer cells (for example with tamoxifen for ESR1-positive breast tumours, PDL1-positive for checkpoint inhibition immunotherapy) or the non-cancer cells (for example PDL1-positive for checkpoint inhibition immunotherapy in gastrointestinal tumours). The latter, for example, may be relevant for future immunotherapies.

The inventors are not aware of any methods in the art that allow deconvolution of cancer cell mRNA expression in single patients. The combined experimental-computational method/algorithm in accordance with the present embodiment requires the physical sectioning of a tumour sample into N parts or sectors. It is understood that methods in accordance with the present embodiment will increase in accuracy with an increase in number, N, of parts or sectors of the tumour sample (for example, for N greater than five to ten). However, it is also understood that some tumour samples may potentially be too small/fragile for such sectioning.

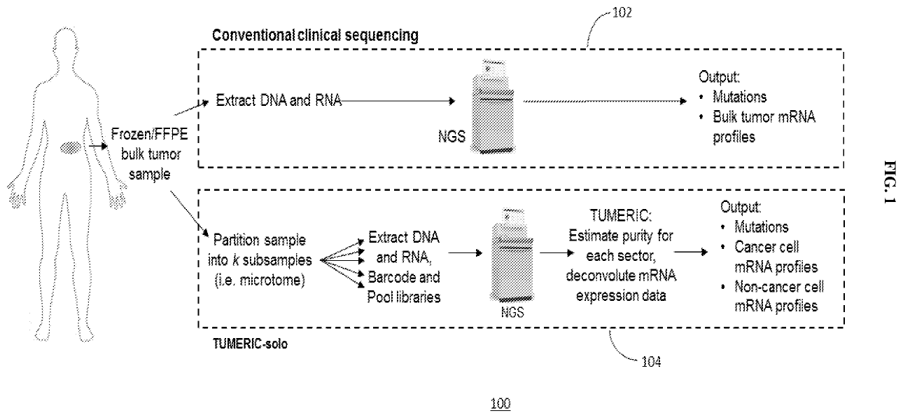

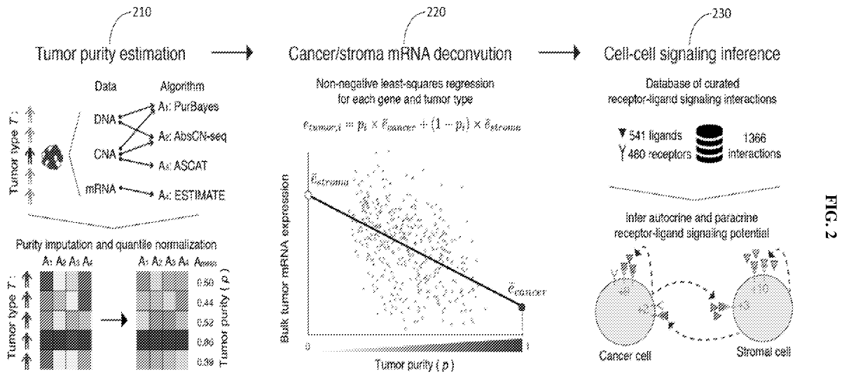

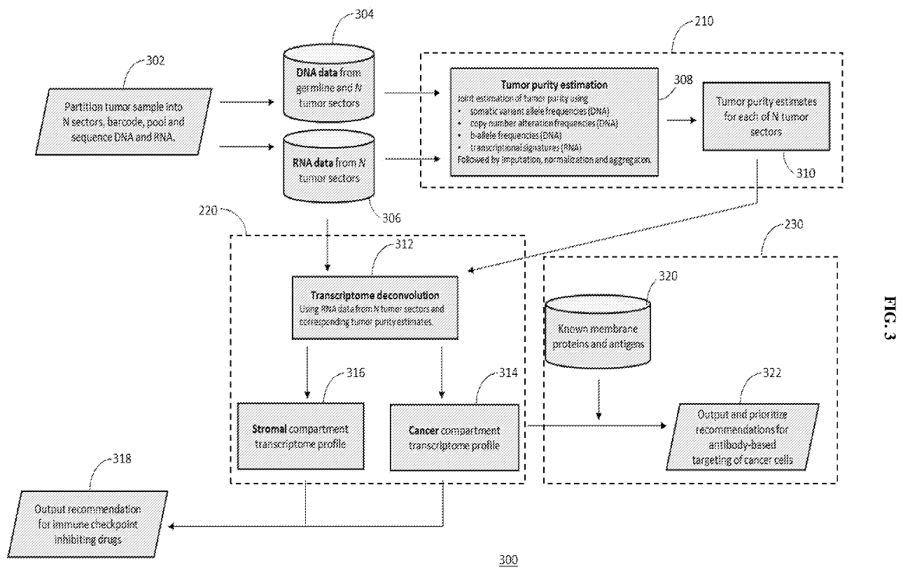

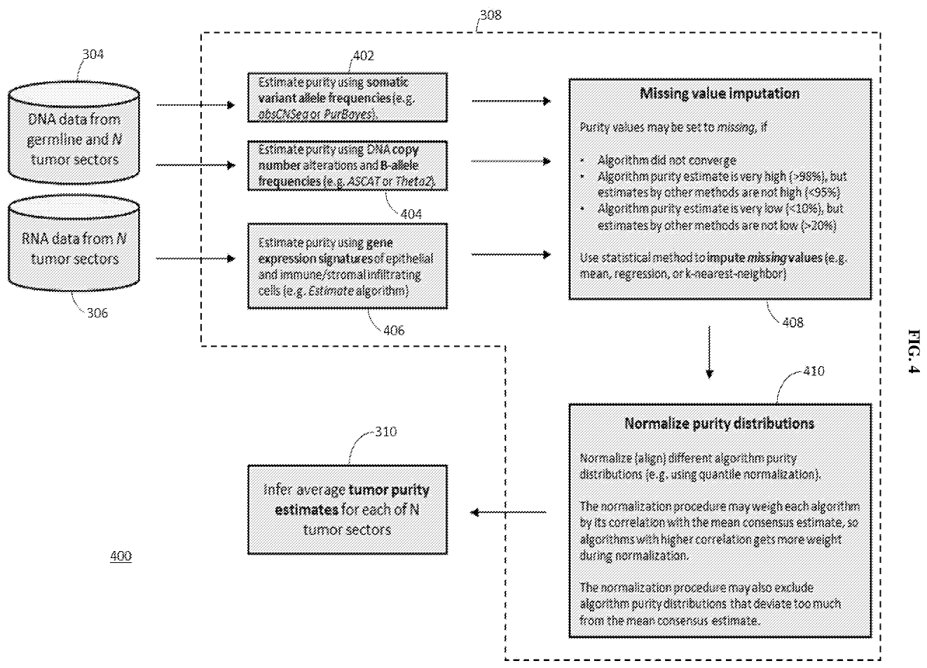

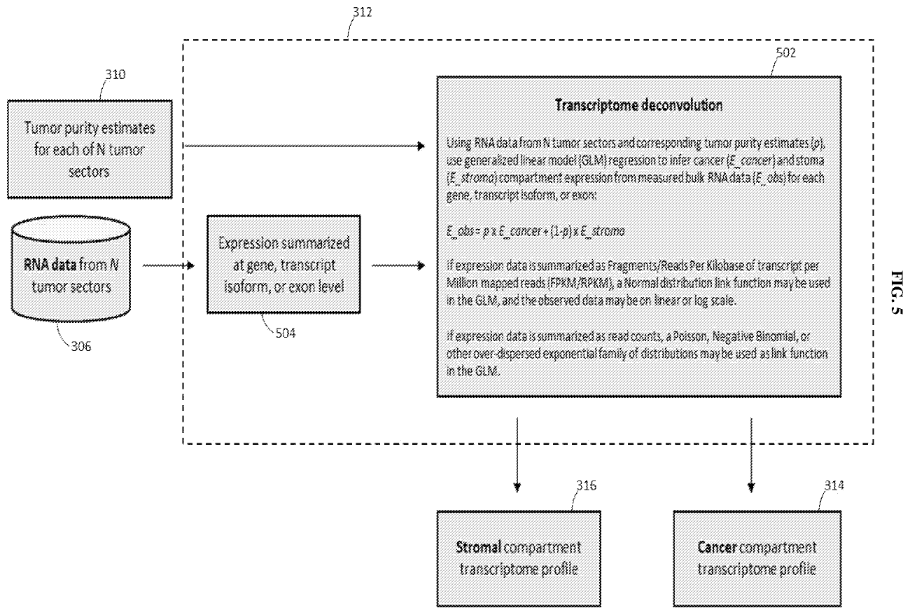

FIG. 1 depicts an illustration 100 comparing operation 102 of conventional clinical sequencing to operation 104 of sequencing in accordance with a present embodiment of TUMERIC-solo. As an alternative to deconvolution using transcriptional signatures, the cancer cell fraction (tumour purity) is first estimated from the mutation allele frequency and copy number profiles of the tumours and averaged to form a consensus tumour purity value. Importantly, the present embodiment avoids making assumptions about the transcriptional profiles of cancer and stromal cells found in a given tumour (see also FIG. 23).

Examples of procedures for estimating tumour purity from DNA and CNA data can for example be found in the following publications: Bao, L., Pu, M., and Messer, K. AbsCN-seq: a statistical method to estimate tumor purity, ploidy and absolute copy numbers from next-generation sequencing data. Bioinformatics 30, 181056-1063; Larson, N., and Fridley, B. PurBayes: estimating tumor cellularity and subclonality in next-generation sequencing data. Bioinformatics 29, 1888-1889. Estimation of purity from gene expression data is shown in the following publication: Yoshihara, K., Shahmoradgoli, M., Martinez, E., Vegesna, R., Kim, H., Torres-Garcia, W., Treviho, V., Shen, H., Laird, P. W., Levine, D. A., et al. (2013). Inferring tumour purity and stromal and immune cell admixture from expression data. Nature Communications 4, 2612.

Thus, in one example, the method disclosed herein predicts expression profiles of cancerous and non-cancerous cells, respectively, based on multiple sets of expression profiles, wherein each set of the multiple sets of expression profiles is obtained from tumour-derived samples comprising a mixture of cancerous and non-cancerous cells of one tumour type. In another example, the method disclosed herein comprises the steps of determining tumour purity values for the one or more tumour-derived samples; providing different sets of expression profiles, wherein the sets of expression profiles comprise combined expression data for multiple or all molecules expressed by cancerous and non-cancerous cells comprised in the one or more tumour-derived samples; and deconvoluting each combined expression data obtained by the method disclosed herein by extrapolating expression profiles of the multiple or all molecules expressed in the different tumour samples with different tumour purity values to a tumour purity value at least substantially equal to 1 or 0; thereby predicting the expression profiles of the cancerous and non-cancerous cells, respectively, from the sets of expression profiles.

In one example, the molecules can be, but are not limited to genes, DNA, RNA or protein molecules, or combinations thereof.

In another example, the method disclosed herein can further comprise scoring molecules disclosed herein based on the level of up-regulation or down-regulation in cancer tissue versus stromal tissue; and/or scoring molecules disclosed herein based on the level of up-regulation or down-regulation in cancer tissue versus healthy tissue.

In another example, the method disclosed herein comprises assigning the up- and down-regulated molecules to genes or transcript isoforms of known data sets of membrane associated proteins or receptors; and/or assigning the up- and down-regulated molecules to genes or transcript isoforms of known data sets of HLA-binding peptides and T-cell antigen binding peptides.

In one example, the known data sets for assigning genes or transcript isoforms originate from, for example and not limited to, Gene Ontology, the Human Protein Atlas, and/or TANTIGEN.

In another example, the gene or transcript isoform disclosed herein can be, but is not limited to, a membrane associated protein, membrane associated receptor, antigen peptide, target protein, peptide, and/or is targetable by an antibody.

When combined with large-scale genomic and molecular data (e.g. from the TCGA or a clinical trial) from human tumours, sequencing in accordance with the present embodiment allows estimation of cancer specific molecular profiles (mRNA, epigenetic, or protein abundance) for target and biomarker discovery using bulk human tumour tissue.

In one example, providing different sets of expression profile comprises the use of existing data sets of expression profiles. In such instances, the existing data sets of expression profiles are from databases such as, but not limited to, TCGA, Genomic Data Commons, cBioPortal, and/or ICGC databases.

Tumour molecular profiles have been deconvoluted into a cancer and stromal cell component using a constrained linear regression approach as described in the TUMERIC-solo sequencing 104 and as described in more detail hereinbelow. To infer autocrine and paracrine signalling crosstalk between these two compartments in the tumour microenvironment (TME), the inferred cancer and stromal compartment expression profiles are combined with curated databases of ligand receptor interactions.

While new computational methods allow inference of cell-type proportions from bulk tumour mRNA profiles using knowledge of primary cell type transcriptional signatures, conventional implementations of these methods generally focus on deconvolution of specific immune cell types and do not provide estimates of gene expression in individual cell types. Previous approaches to estimate cancer and stromal cell gene expression profiles in tumour tissue have either been strongly customized for individual tumour types or have assumed the tumour to be a mixture of cancer cells and healthy tissue. Individual tumour cell customization restricts the use of such methods, and assuming that the tumour is a mixture of cancers cells and healthy tissue ignores the unique stromal cell types and biological processes of the tumour microenvironment, which may strongly confound the inferred gene expression profiles.

Few experimental techniques exist that allow discrimination between signals from cancer and non-cancer cells in the tumour microenvironment. Immunohistochemistry (IHC) can directly measure selected proteins in tumour tissue but is generally not quantitative and not suited for large-scale and unbiased profiling or discovery. Furthermore, IHC is labour intensive and requires a trained pathologist to aid the data interpretation.

Transcriptome-wide profiles of cancer and stromal cell may be generated using micro-dissection or single-cell profiling of tumour tissue, but these approaches are difficult to apply to tumour biopsies, and disassociation may to some extent also confound cell physiology and gene expression profiles. Furthermore, these methods require special handling and processing of the tissue, which makes them less suited as standard data generation assays in precision oncology.

Targeted exome sequencing is becoming a routine diagnostic assay with companies offering clinical sequencing as a service. See, for example, FIG. 20. Due to the continued drop in sequencing cost, companies are now also offering whole exome and RNA sequencing as a clinical diagnostic service. Importantly, these services are scalable because they require only frozen or Formalin-Fixed Paraffin-Embedded (FFPE) tumour tissue and next-generation sequencing (NGS). However, bulk Exome and RNA sequencing does not allow direct measurements of cancer cell population in tumours. This is important, for example, to determine breast cancer patients with estrogen positive tumours (for tamoxifen treatment) or tumours with increased PDL1 expression in cancer cells (PD1/PDL1 checkpoint inhibition).

TUMERIC is a method which estimates cancer and stromal (comprising any non-cancer cell) compartment molecular profiles, and cross-talk signalling between average representative cells in these two compartments, for a set of tumours. Referring to FIG. 2, an overview illustration 200 of the TUMERIC sequencing process in accordance with the present embodiment begins with tumour purity estimation 210. The purity (fraction of cancer cells) of each bulk tumour sample is estimated 210 from DNA (Exome-sequencing), copy number (aCGH), and mRNA expression (RNA-sequencing) data using a consensus approach. Next, deconvolution 220 of mRNA expression levels in “average” cancer and stromal cells are inferred for a given gene and a set of tumours (e.g. representing a tumour type) using non-negative least-squares regression. Finally, using a database of curated receptor-ligand signalling interactions, the derived mRNA expression profiles are used 230 to infer candidate autocrine and paracrine signalling pathways between cancer and stromal cells.

Thus, in one example, the method disclosed herein can comprise, but is not limited to, determining the tumour purity value based on, but not limited to, distribution of somatic DNA variant allele frequencies, somatic DNA copy number alteration amplitudes, germline B-allele frequencies, gene expression signatures or patterns, protein expression signatures or patterns, and DNA methylation signatures or patterns, and combinations thereof. In one example, the tumour purity value is based on gene expression signatures (or gene expression profiles). In another example, the tumour purity value is based on allele frequencies, for example, somatic DNA variant allele frequencies and/or germline B-allele frequencies. In another example, the tumour purity value is based on methylation signatures.

In one example, at least two, or at least three, or at least four, or at least five, or two, or three, or four, or five or all of the methods disclosed herein are used together to determine mean tumour purity.

In another example, the tumour purity value is a mean tumour purity value.

In one example, the tumour type referred to herein can be, but is not limited to, BLCA, BRCA, CESC, CRC (COAD and READ combined), ESCA, GBM, HNSC, KIRC, KIRP, LGG, LIHC, LUAD, LUSC, OV, PAAD, PRAD, SKCM, STAD, THCA and UCEC, as referenced in the TCGA database.

Referring to FIG. 3, a flow diagram 300 discloses the TUMERIC-solo method in accordance with the present embodiment. Initially, a frozen tumour sample is partitioned 302 into N sectors (for example, where the value of N is larger than five but less than twenty (5<N<20)), using for example a microtome, cryosectioning, or frozen tumour arrays. DNA data and RNA data are simultaneously extracted from each sector, bar-coded, pooled, and profiled with next-generation sequencing. The obtained next-generation DNA sequencing data 304 and the obtained next-generation RNA sequencing data 306 is de-multiplexed, and the DNA sequencing data 304 and the RNA sequencing data 306 is used to estimate 308 the cancer cell fraction (tumour purity) 310 for each sector using mutation allele frequencies and copy number profiles and transcriptional signatures of each sector. The purity data 310 (pᵢ) and the sector-wise RNA sequencing data 306 (E_{tumor,i}) is deconvoluted 312 as discussed hereinbelow and as shown in Equation (1) (see also FIG. 21) to infer molecular profiles of cancer cells 314 (E_{cancer}) and non-cancer cells 316 (Eₛₜᵣₒₘₐ) in the original tumour sample.

E_{tumor,i}=pᵢ×E_{cancer}+(1−pᵢ)×Eₛₜᵣₒₘₐ (1)

The profiles of cancer cells 314 and non-cancer cells 316 can be used to provide recommendations 318 for immune checkpoint inhibiting drugs. In addition, cross-referencing with a database 320 of known membrane proteins and antigens cell-cell signalling can be can be used to determine and prioritize recommendations 322 for antibody-based targeting of cancer cells from the cancer cell profiles 314.

Referring to FIG. 4, a flow diagram 400 depicts the TUMERIC-solo tumour purity estimation process 308 in accordance with the present embodiment. The purity of each bulk tumour sample (i.e., the fraction of cancer cells in each sample) is inferred by first estimating purity from the DNA sequencing data 304 and the RNA sequencing data 306 using three methodologies. Purity is estimated from the DNA sequencing data 304 using somatic variant allele frequencies 402 and using DNA copy number alterations and B-allele frequencies 404, and purity is estimated from the RNA sequencing data 306 using gene expression signatures of epithelial and immune/stromal infiltrating cells 406. If any of the estimation methodologies 402, 404, 406 do not converge or result in estimations too high or too low, the purity value estimation is imputed 408 using a statistical method (e.g., mean, regression, or k-nearest neighbour) for imputation. An estimation is deemed to be too high when the estimate by one of the three methodologies 402, 404, 406 is very high (e.g., >98%), but the estimates of the other methodologies 402, 404, 406 are not as high (e.g., <95%). Similarly, the estimation is deemed to be too low when the estimate by one of the three methodologies 402, 404, 406 is very low (e.g., <10%), but the estimates of the other methodologies 402, 404, 406 are not as low (e.g., >20%). The final step in the tumour purity estimation 308 to infer 310 the average tumour purity estimates for each of the N tumour sectors is normalization 410 of the purity distributions. The normalization 410 aligns different estimated purity distributions. This may be performed by using quantile normalization or other normalization techniques and/or by weighing each estimation by its correlation with a mean consensus estimation so that estimations with higher correlation to the mean consensus estimation are weighted higher during normalization. The normalization 410 may also exclude purity estimation distributions that deviate too much from the mean consensus estimation.

Thus, in one example, the tumour-derived sample is obtained from a single subject. In another example, the tumour-derived sample is partitioned into 2 or more sections. In yet another example, the tumour-derived sample is partitioned into 2 or more sections, and wherein one set of expression profiles is generated for each section.

Referring to FIG. 5, a flow diagram 500 depicts the TUMERIC-solo transcriptome deconvolution 312 in accordance with the present embodiment. The purity data 310 (i.e., the tumour purity estimates for each of the N tumour sectors) and the sector-wise RNA sequencing data 306 is deconvoluted 312 to infer molecular profiles of cancer cells 314 and non-cancer cells 316 in the original tumour sample. The deconvolution 312 includes transcriptome deconvolution 502 of the tumour purity estimates 310 and the RNA sequencing data 306 having its expression summarized 404 at a gene, a transcript isoform or an exon level.

In one example, the expression profiles can be, but are not limited to, gene expression, RNA expression, epigenetic expression, protein expression, proteomic expression, and combinations thereof, for example, RNA and epigenetic expression, and RNA and protein expression. In another example, the expression profiles are gene expression profiles. In another example, the expression profiles are RNA expression profiles.

Using the tumour purity estimates (p) 310 and the RNA sequencing data 306, the transcriptome deconvolution 402 advantageously uses a generalized linear model (GLM) regression to infer cancer (E cancer) compartment expression 314 and stroma (E_stroma) compartment expression 316 from the measured bulk RNA data (E_obs) 306 for each gene level, transcript isoform level or exon level at which the RNA data is summarized 404 as shown in Equation 2:

E_obs=(p×E_cancer)+((1−p)×E_stroma) (2)

If the expression data 314, 316 is summarized as fragments/reads per kilobase of transcript per million mapped reads (FPKM/RPKM), a normal distribution link function may be used in the generalized linear model (GLM) in accordance with the present embodiment and the observed data may be on a linear or a log scale. If the expression data 314, 316 is summarized as read counts, a Poisson, a Negative Binomial, or other over-dispersed exponential family of distributions may be used as the link function in the generalized linear model (GLM) in accordance with the present embodiment.

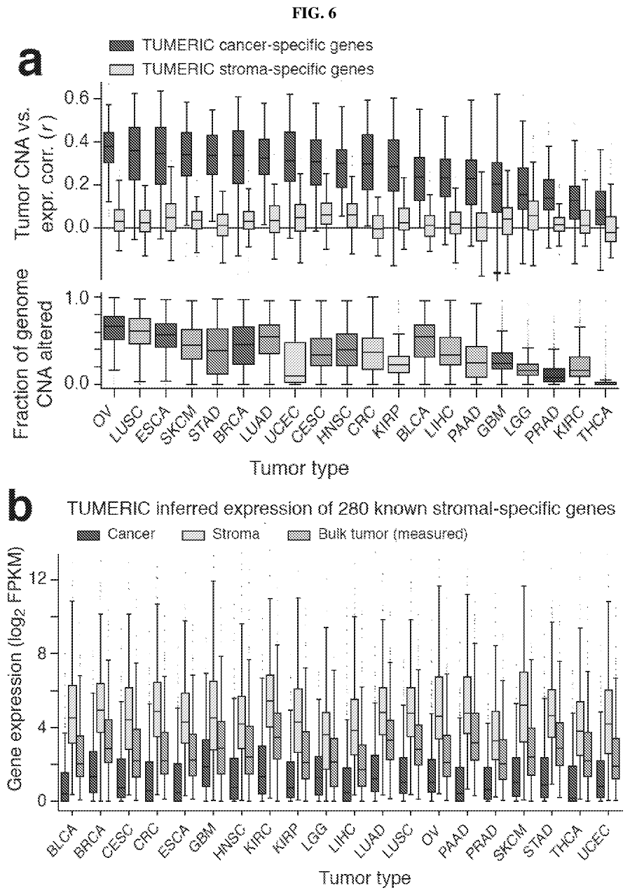

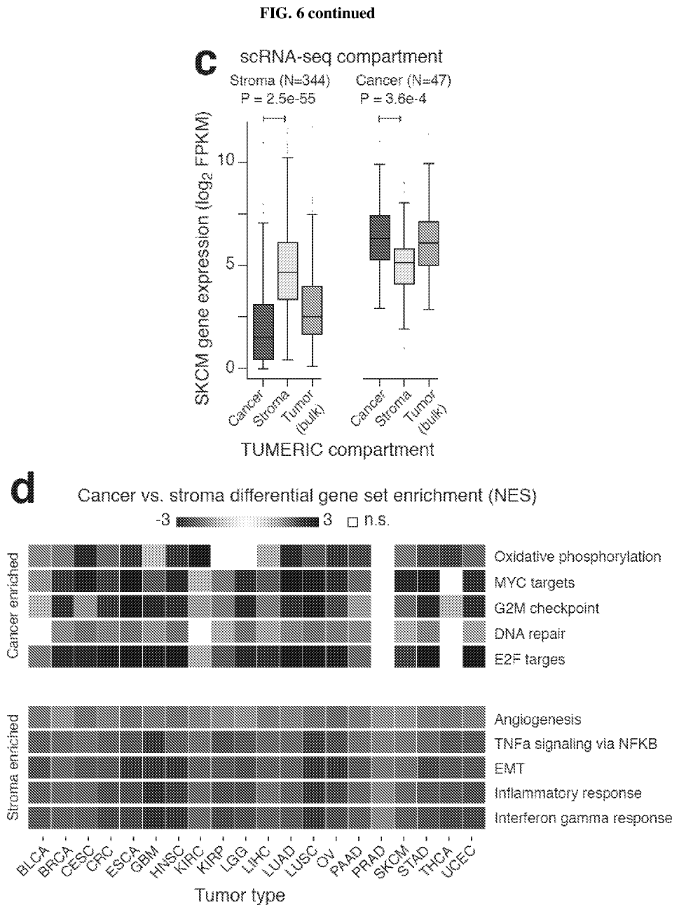

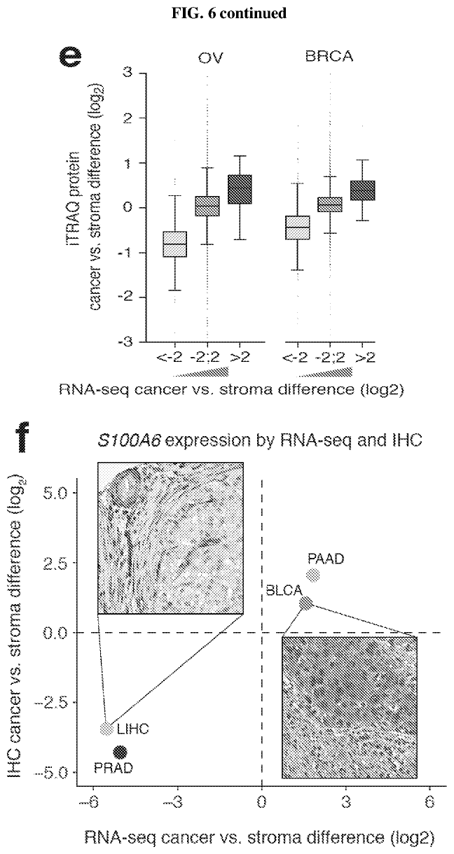

FIG. 6 depicts a working example validating the tumour transcriptome deconvolution in accordance with the present embodiment wherein, as shown in FIG. 6a, consensus tumour purity estimates are derived for about 8000 samples across 20 solid tumour types in the Cancer Genome Atlas (TCGA) and shows that most tumour samples had a purity of 40-70%. Pancreatic adenocarcinoma (PAAD) tumours were shown to have very low purity (mean purity ˜39%), consistent with previous observations. The glioblastoma (GBM) and ovarian cancer (OV) cohorts had the highest purity estimates, likely due to tumour selection bias in the first phase of the Cancer Genome Atlas project. It was found that mRNA expression-derived tumour purity estimates, as well as previously published consensus tumour purity estimates, were well correlated with TUMERIC consensus purity estimates, but were likely systematically overestimating purity by 20-50% compared to mutation and copy number based methods (FIG. 35).

FIG. 6b shows genes specifically expressed in cancer and stromal cells that were inferred for each tumour type. The correlation between mRNA expression and somatic copy number alterations (CNA) at each gene locus was evaluated (top panel). Tumour types were ordered by the difference in means of cancer and stromal gene correlations and the fraction of genome altered by CNA was determined for each tumour sample (bottom panel). Multiple analyses were performed to evaluate the accuracy of TUMERIC in deconvoluting cancer and stromal cell compartment transcriptomes. Firstly, since somatic copy number alteration (CNA) is a hallmark of cancer cell genomes, without being bound by theory, it was reasoned that expression of genes deriving exclusively from stromal cells should not be affected by such alterations. Indeed, using TUMERIC to infer the top cancer and stromal-cell specific genes in each tumour type, a strong correlation was found between tumour copy number alteration and expression of cancer-specific genes, but not between tumour copy number alteration and expression of stroma-specific genes. Variations in correlation between tumour types could be explained by the overall prevalence of copy number alterations in a given tumour type. Similarly, it was found that previously derived stromal and immune cell-specific genes were consistently inferred by TUMERIC to have markedly higher expression in the stroma compartment of all tumour types as shown in FIG. 6c where the inferred cancer and stroma compartment expression levels for 280 known stromal-specific genes are depicted.

To test the concordance of TUMERIC with tumour single-cell RNA-sequencing (scRNA-seq) profiling, TUMERIC expression estimates for cancer and stromal cell-specific genes identified by single-cell RNA-sequencing of melanoma tumours were compared. FIG. 6d shows the inferred cancer and stroma compartment expression levels in melanoma (skin cutaneous melanoma—SKCM), as well as bulk tumour measurements, for cancer and stroma specific genes previously identified with melanoma tumour single cell RNA sequencing (scRNA-sequencing). TUMERIC inferred significantly higher stroma-compartment expression for stromal-cell specific genes (P=2e-55, Mann Whitney, two-tailed), and significantly higher cancer-compartment expression for cancer-cell specific genes (P=3.6e-4).

The likely biological function of genes with cancer or stroma-specific expression across tumour types was evaluated using gene set enrichment analysis (see methods section). Gene sets consistently up-regulated in cancer compartments across tumour types were associated with known hallmarks of cancer cells such as activation of cell cycle, MYC signalling, metabolism, and DNA repair. FIG. 6e shows genes which are ordered by inferred expression difference between cancer and stroma compartments in each tumour type. Gene set enrichment analysis (GSEA) was used to identify cancer and stroma enriched gene sets. Non-significant associations (false-discovery rate (FDR)>0.25) are displayed in white. In contrast, gene sets consistently up-regulated in stromal compartments across all cancer types included genes related to angiogenesis, immune response, and mesenchymal cell state.

To evaluate the extent that the deconvoluted mRNA profiles represent an accurate proxy for protein levels in the cancer and stromal cells, TUMERIC was applied to deconvolute protein expression data from TCGA tumours. FIG. 6f shows protein expression inferred for cancer and stroma compartments in (OV) and breast (BRCA) cancer cohorts using iTRAQ protein quantification data and compared to RNA sequencing data and it was found that mRNA expression estimates were generally concordant with relative levels of cancer and stroma protein abundance.

Finally, FIG. 6g depicts genes identified with highly variable cancer vs. stroma mRNA expression differences across cancer types and immunohistochemistry (IHC) staining data was compared to RNA sequencing data for the gene (S100A6) with highest mRNA abundance to confirm that expression patterns of one such gene was indeed variable across tumour types (FIG. 6g).

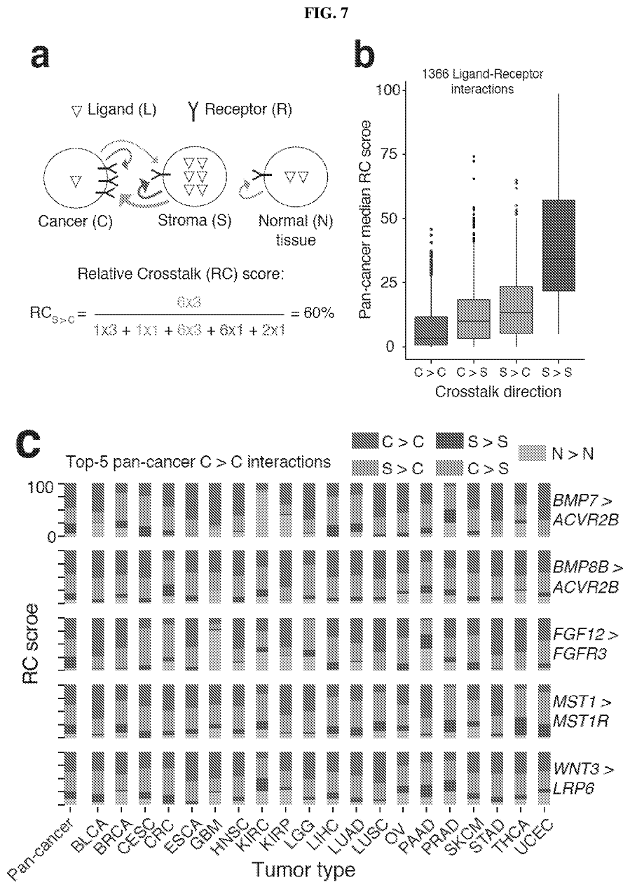

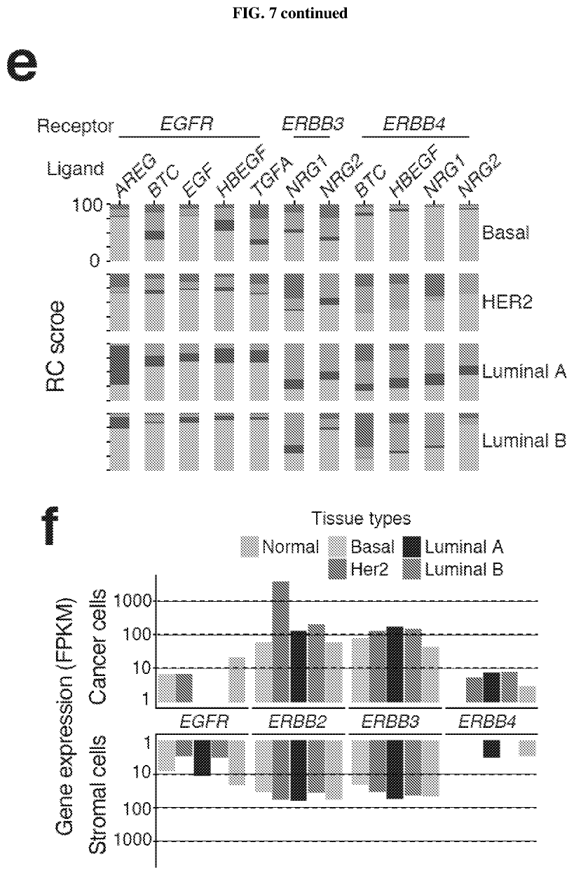

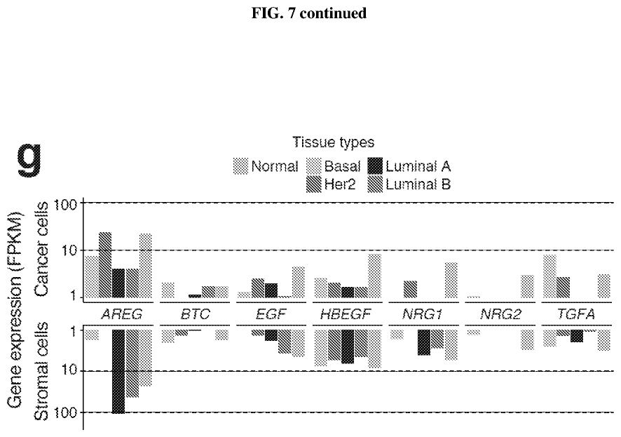

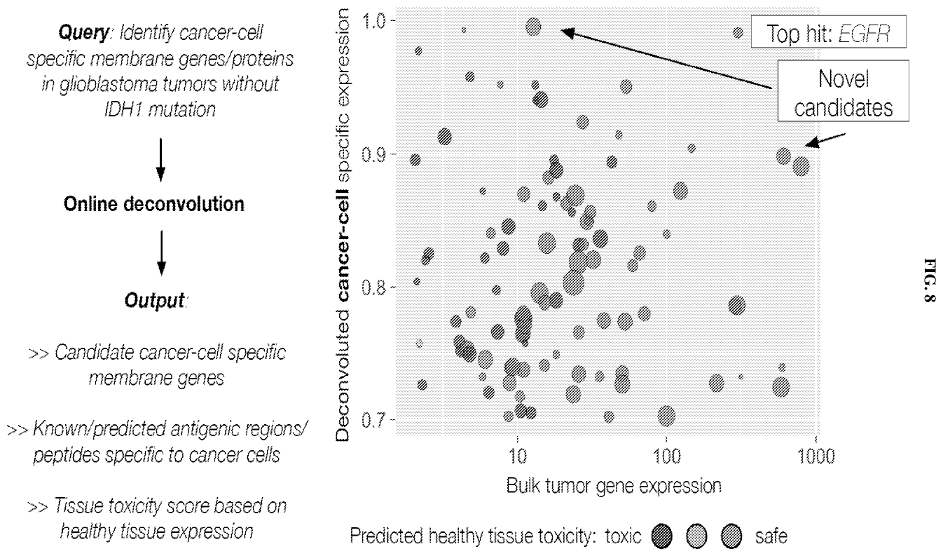

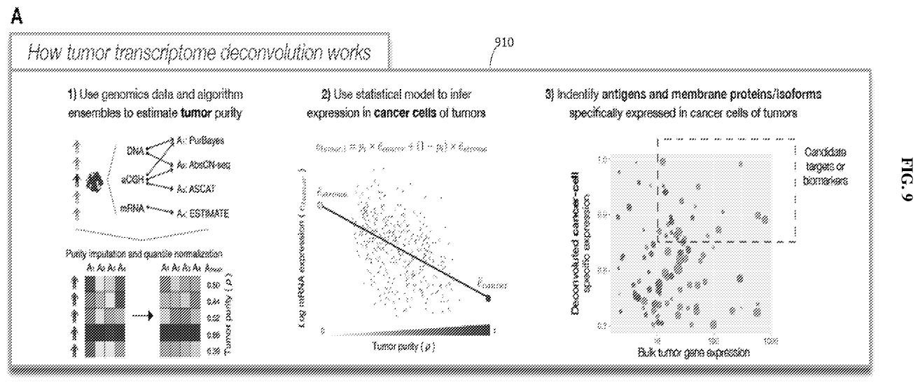

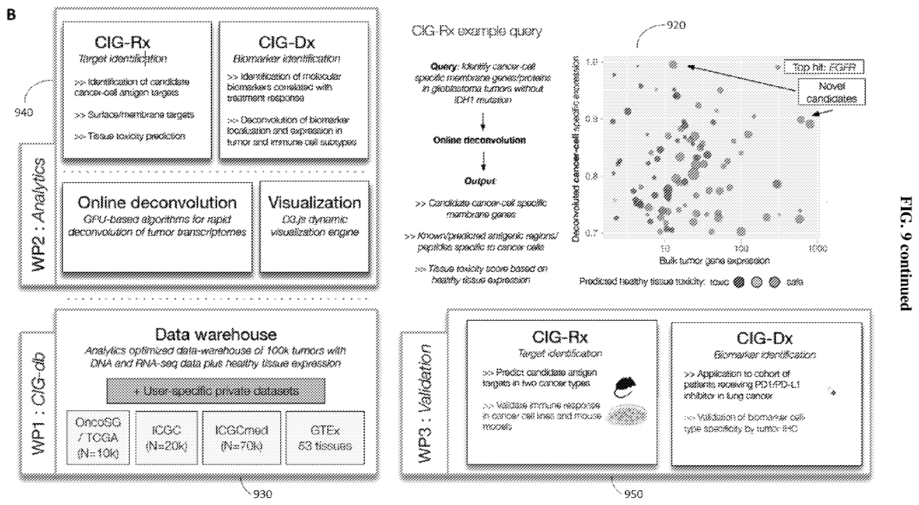

Referring to FIG. 7, the results of the inference of crosstalk between cancer and stromal cells in accordance with the present embodiment are depicted. To infer and differentiate between types of autocrine (signalling within same compartment) and paracrine (signalling between cancer and stromal cell compartment) ligand-receptor (LR) crosstalk within the tumours, a metric, the Relative Crosstalk (RC) score was developed. This Relative Crosstalk (RC) score estimates the relative flow of signalling in four possible directions between cancer and stromal cell compartments, including a bulk (non-deconvoluted) normal tissue signalling estimate and quantifies the relative signalling directionality of a given ligand-receptor pair as shown in FIG. 7a, as well as making multiple simplifying assumptions about cell-cell signalling (e.g. ignoring local competition and saturation effects). Nevertheless, the Relative Crosstalk (RC) score is a reasonable approximation to determine the relative signalling directionality in the tumours.

Firstly, the extent to which some ligand-receptor pairs showed consistent modes of crosstalk across tumours types was evaluated, and a difference between Relative Crosstalk scores in the cancer and stromal compartment was found. While only three ligand-receptor pairs showed evidence of strong autocrine cancer signalling across tumour types (median cancer-to-cancer RC score >40%), 264 ligand-receptor pairs were found with high autocrine stroma signalling scores as shown in FIG. 7b. This suggests that, for solid tumours, autocrine cancer signalling tend to be tumour type specific and likely determined by the cancer cell-of-origin, while stromal autocrine signalling is generally independent of tumour type and site of origin. Interestingly, the paracrine signalling interface between cancer and stromal cell compartments also had a high number of recurrent interactions (26 and 40 interactions with median RC score >40% for cancer-to-stroma and stroma-to-cancer signalling, respectively), highlighting the importance of the tumour environment on cancer cell biology. Inferred recurrent autocrine cancer signalling across tumour types involved signalling through FGFR8, LRP6 and MST1R as shown in FIG. 7c. It is of note that MST1R (RON) has been found to be a prognostic marker and it is currently being evaluated as a therapeutic target in a range of tumour types. Signalling through ACVR2B was notably both among the top inferred cancer autocrine and stroma-to-cancer signalling interactions across tumour types (FIGS. 7c and 7d).

By way of another working example, the method disclosed herein was used to analyse ˜130 lung adenocarcinoma tumour samples, all samples had exome (DNA) and RNA sequencing data. A patient tumour sample (A014) that had been partitioned into eight independent sectors and then subjected to the TUMERIC-solo analysis workflow has also been analysed. The methodology in accordance with the present embodiment was further used to study the role of EGF-family signalling across subtypes of breast cancer as shown in FIG. 7e. A 30-fold increased expression of ERBB2 in cancer cells of HER2-positive tumours was inferred as shown in FIG. 7f. Focusing on canonical EGF-family LR interactions and inferred signalling through this receptor, it was found that both cancer and stromal cell EGFR expression was generally lower in tumours compared to normal breast tumours (FIGS. 7e and 7f). EGFR expression was inferred to be expressed in cancer cells of basal and HER2-positive tumours, but near absent in cancer cells of Luminal A and B tumour subtypes (FIGS. 7e and 7f). Amphiregulin (AREG) appeared to be a major source of EGFR ligands (FIG. 7g). It is of note that while AREG was inferred to be predominantly expressed by stromal cells in both Luminal subtypes, AREG was expressed almost exclusively by cancer cells in basal and HER2-positive tumours (FIG. 7g). This data supports the presence of a cancer-cell autocrine feedback loop between AREG and EGFR that is unique to HER2-positive and basal breast tumours, and demonstrates how this approach can be applied to study cell-cell crosstalk associated with specific molecular or genetic subtypes of tumours.

In summary, provided herein is a data-driven method to deconvolute cancer and stromal cell transcriptomes and estimate cell-cell crosstalk in the tumour microenvironment using only bulk genome and transcriptome data from a set of tumours. The method disclosed herein is not restricted to transcriptomic data, and can advantageously be used with other types of bulk tumour molecular data such as, but not limited to, epigenetic or proteomic profiles.

### Validation of TUMERIC-Solo Approach

First, the ability of TUMERIC and TUMERIC-solo to quantify cancer and stroma expression for known marker genes was evaluated. Referring to FIG. 8, this figure shows an example query to illustrate the process of identifying membrane protein drug targets in glioblastoma tumours using TUMERIC. In this query, the user specifies the tumour type (Glioblastoma) and further specifies a genetic/molecular subtype of tumours to analyse (here tumours without IDH1 mutations). Known membrane proteins are then ranked by their overall bulk tumour expression (x-axis) and the extent, as inferred by TUMERIC, that they are expressed specifically in cancer cells (y-axis). Predicted toxicity of each target, e.g. derived from gene expression in healthy vital organs such as brain/heart/kidney, can be co-visualized and aid in the target selection process.

Referring to FIG. 9A, a schematic illustration 910 represents an outline of tumour transcriptome (or proteome) deconvolution methodologies and platforms in accordance with the present embodiment. FIG. 9B depicts work packages WP1 920, WP2 930 and WP3 940 along with an overview 950 of the methodology in accordance with the present embodiment.

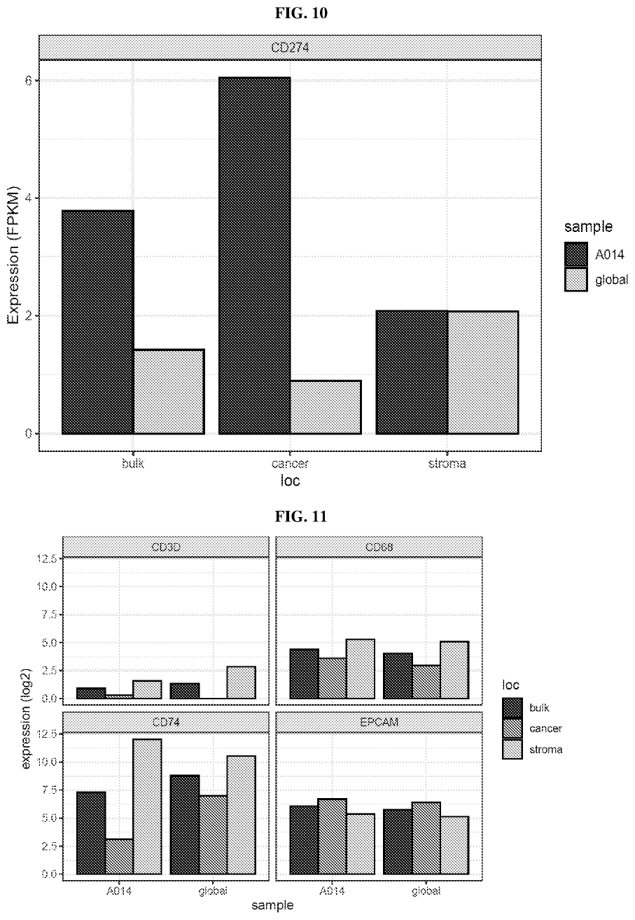

Referring to FIG. 11, bar graph data from TUMERIC-Solo is depicted as applied to a single lung cancer patient (A014) as compared to data from a cohort of patients (TUMERIC applied to about 60 lung cancer patients). Firstly, this data demonstrates that TUMERIC-Solo can reliable identify known stromal factors (overexpressed in stroma compared to cancer, such as CD3D, CD68) and epithelial/cancer markers (EGFR, EPCAM). Secondly, using TUMERIC-Solo it was shown that PDL1 (CD274) expression is generally expressed in the stroma, but in patient A014 PDL1 it is overexpressed more than six-fold (>6 fold) in cancer cells, while stroma expression remains unchanged (FIG. 10). This identifies PD1/PDL1 checkpoint blockade as a potential target in this specific patient. Performing the same analysis using bulk tumour profiling, one would observe an about two-fold up-regulation in PDL1 expression, whereby it would be unknown if the increase in expression level is due to stroma or cancer cells overexpressing PDL1.

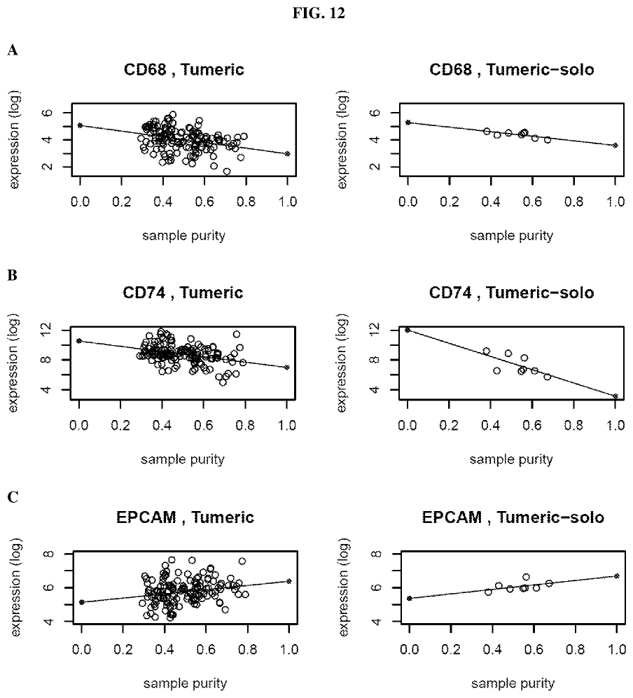

This demonstrates how TUMERIC and TUMERIC-solo can yield concordant results, even though TUMERIC uses data obtained from different patient tumours and TUMERIC-solo uses data from different sections of one individual patient tumour. To further illustrate this concept and concordance, the two deconvolution approaches were illustrated by plotting the measured (bulk) gene expression of CD68, CD74, and EPCAM as a function of estimated sample/sector tumour purity for TUMERIC (N=130 samples) and TUMERIC-solo (N=8 sectors of patient tumour A014), respectively (FIG. 12). While the analysis and inferred gene expression levels are overall concordant between TUMERIC and TUMERIC-solo, the analysis of CD74 demonstrates how TUMERIC-solo can infer patient-specific changes in gene expression.

### Inferring Patient-Specific PDL1 Expression Using TUMERIC-Solo

Tumour PDL1 (CD274) expression is a biomarker of immune checkpoint inhibition treatment response in lung cancer. However, PDL1 checkpoint inhibition only works in a subset of patients (<20%), and whether it is cancer or stromal cells that predominantly over-express PDL1 in the patients benefitting from treatment is being debated. TUMERIC-solo analysis of the A014 tumour identified that PDL1 was highly up-regulated in cancer cells, but not in stromal cells. Of note, PD-L1 up-regulation was a A014 patient-specific phenomenon, and was not observed with TUMERIC analysis of the 130 patient tumours, highlighting the added value of TUMERIC-solo. In summary, this indicates that PD1/PDL1 immune checkpoint inhibition could be an effective treatment for patient A014. Furthermore, the signal-to-noise ratio (SNR) for TUMERIC-solo (six cancer vs one background/global) was much higher than a naive bulk tumour (3.9 bulk vs. 1.7 background/global) measurement of PDL1 up-regulation (FIG. 10).

Improved Quantification of Immune Checkpoint Biomarker Signature with TUMERIC-Solo

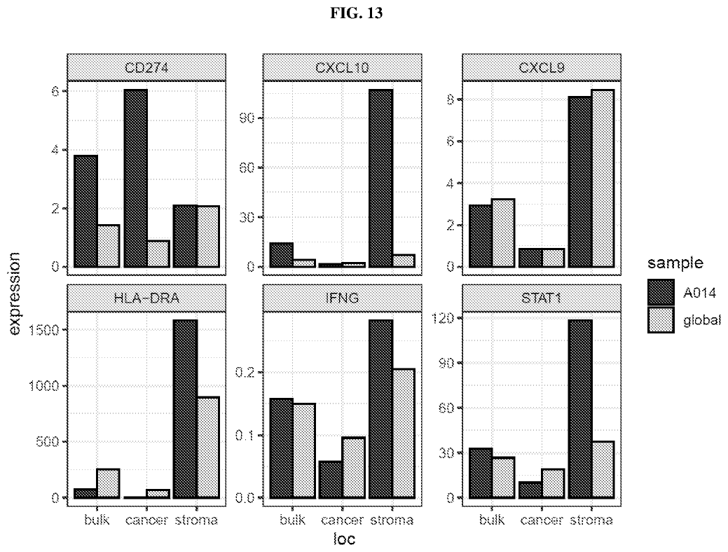

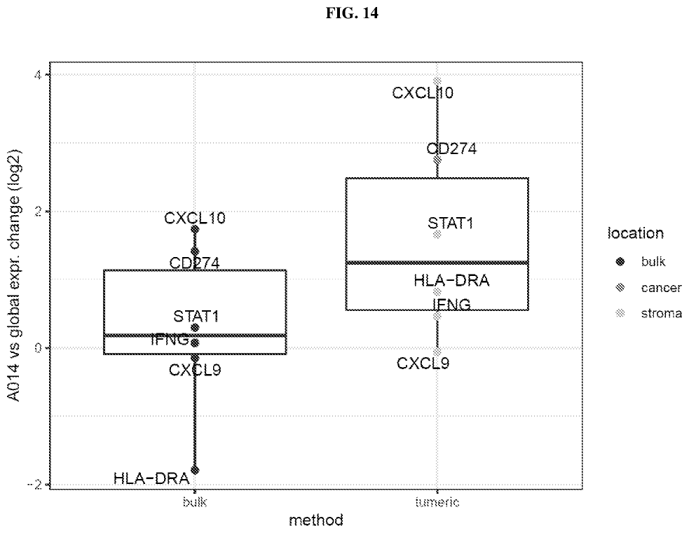

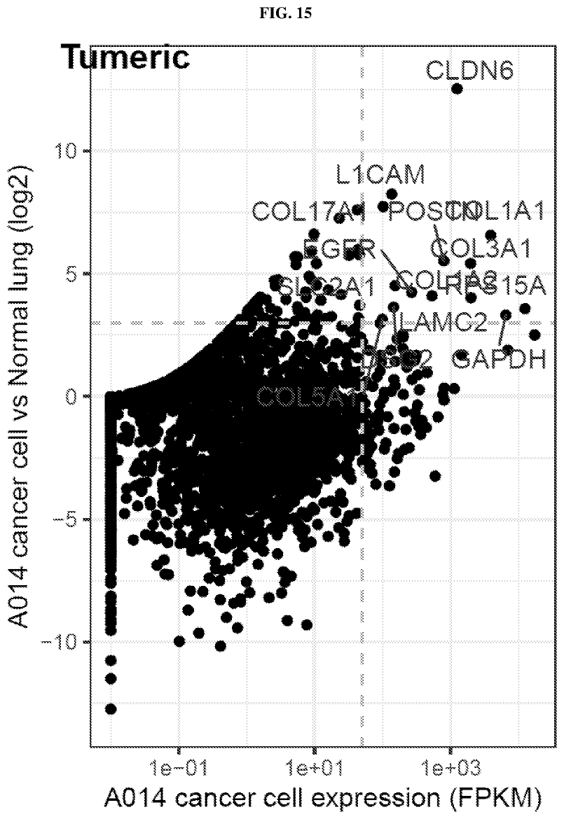

It had been previously reported that a bulk tumour 6-gene biomarker was responsible for response to pembroluzimab (PD1/PDL1 inhibition) treatment. These six genes are IDO1/CD274, CXCL10, CXCL9, HLA-DRA, STAT1, and IFNG. TUMERIC-solo was used to infer the activity of these genes in patient A014. This analysis demonstrated that one gene was strongly up-regulated in cancer cells (CD274/PDL1), while four other genes strongly up-regulated in the stroma (CXCL10, HLA-DRA, IFNG, STAT1) (FIG. 13). The signal-to-noise ratio for TUMERIC-solo and a naive bulk tumour approach was compared for the combination of these 6 markers. It was found that TUMERIC-solo provided a marked improvement in signal-to-noise ratio for these markers due to its ability to distinguish between cancer and stroma expression (FIG. 14). TUMERIC-solo may therefore provide a more accurate aggregated biomarker activity score for recommendation of pembroluzimab treatment.

### Using TUMERIC and TUMERIC-Solo to Guide Therapy and Target Discovery

TUMERIC and TUMERIC-solo can be applied to sets of patient tumours or an individual tumour to identify and/or nominate drug targets and treatments as seen in FIGS. 8 and 9 above. An outline of the methodology in accordance with the present embodiment is disclosed herein, using at least the following steps: 1. Apply TUMERIC/TUMERIC-solo to set of samples/sectors; 2. Rank genes or transcript isoforms by inferred cancer compartment expression; 3. Score genes or transcript isoforms by level of up-regulation in cancer vs stromal compartment (identify cancer-cell specific factors); 4. Score genes or transcript isoforms by level of up-regulation in cancer vs healthy/normal tissue (identify cancer-cell specific factors); 5. Subset genes or transcript isoforms to known membrane-associated proteins or receptors (using, for example known resources/databases). This will yield a shortlist of targets for antibody based (e.g. Antibody drug-conjugates) therapy; 6. Subset genes or transcript isoforms of proteins generating known HLA-binding and T-cell antigen peptides (using, for example known resources/databases). This will yield a shortlist of tumour associated antigens (TAAs) specifically associated with and overexpressed in the cancer cells of the tumour(s), nominating candidates for engineered T-cell based therapies (such as, but not limited to CAR-T).

Thus, in one example there is disclosed a method of analysing a single patient tumour. The method disclosed herein is also capable of identifying aberrantly expressed transcripts in cancer cells of a single patient. The method disclosed also allows unbiased analyses to be performed requiring only a minimum number of (mathematical) assumptions.

Patient Specific Recommendation of Therapeutic Antibodies with TUMERIC-Solo

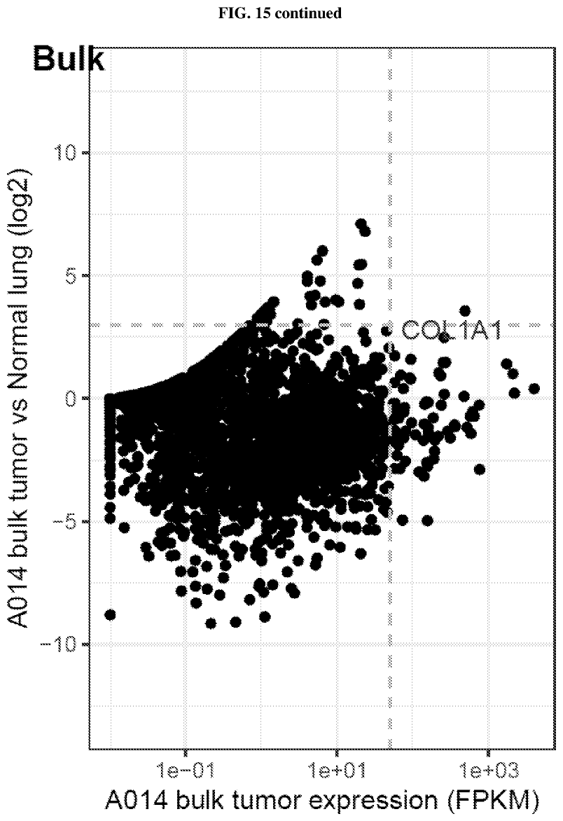

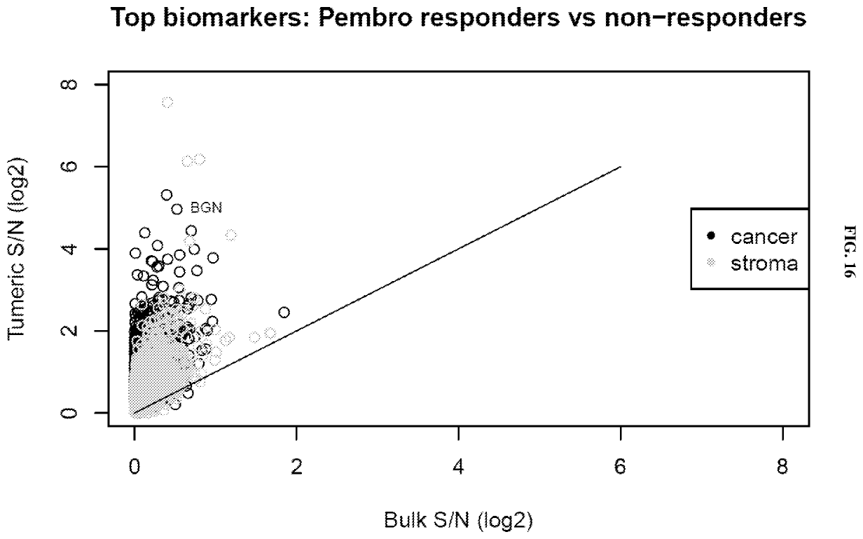

The extent to which the method disclosed herein (TUMERIC-solo) could be used to make a recommendation with regard to treatment with specific antibodies targeting membrane proteins of cancer cells was analysed in subject A014. About 4000 known and annotated membrane proteins for specific (log fold-change >3, cancer vs normal lung) and abundant expression (expression >50 FPKM) in cancer cells of the A014 tumour were analysed, as these are parameters that are critical for a therapeutic antibody target. The top target with this approach using TUMERIC-solo was CLDN6, which is currently being evaluated as a therapeutic antibody target elsewhere (FIG. 15). Thus, the result is that TUMERIC-solo indicates that targeting CLDN6 in patient A014, for example by using a therapeutic antibody against CLDN6, is recommended. A similar naive bulk target recommendation approach only highlighted a single target (COL1A1), and failed to report the CLDN6 antibody target.

### TUMERIC-Solo Reveals Biomarkers of PD-L1 Inhibition Treatment Response in Gastric Cancer

It was further tested whether TUMERIC or TUMERIC-solo could reveal previously untargeted biomarkers of PD-L1 inhibition treatment response by estimating gene expression more specifically in cancer or stromal/immune cells (as compared to bulk tumour tissue). In this regard, TUMERIC is used to identify robust biomarkers across a cohort of treated patients, and TUMERIC-Solo is then applied as a biomarker test assay (companion diagnostic) in the setting of treating an individual patient. Data from a recent cohort of about 50 metastatic gastric cancer patients treated with a PD-L1 inhibitor (pembrolizumab) was used. The patients were divided into groups based on their treatment response (complete/partial response (R); stable disease (SD); progressive disease (PD)), and TUMERIC was applied within each group of patients.

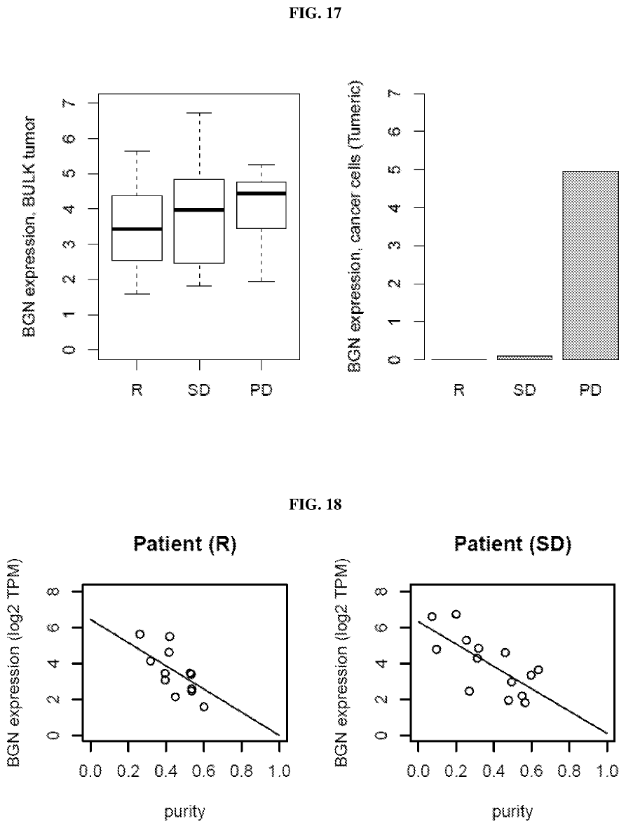

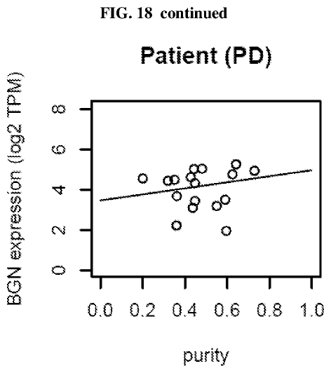

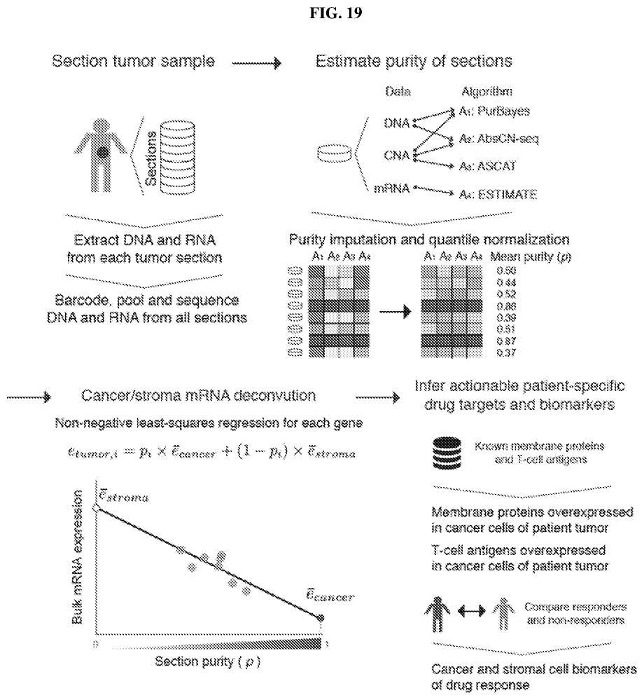

Firstly, this analysis revealed a large set of genes with robust cancer or stromal cell gene expression dysregulation between responders (R) and non-responders (PD). The signal-to-noise ratio (predictive power) for these genes were much stronger with TUMERIC than when measured through bulk tumour profiling (see FIG. 16), suggesting that many of these biomarkers would only be useful in combination with TUMERIC-solo. For example, biglycan (BGN) shows a very high expression level in cancer cells of non-responding patients (PD), but a near-zero expression level in responding patients (R+SD). This difference is much less pronounced, and more variable, with bulk tissue gene expression profiling (See FIG. 17); a test based on bulk BGN expression alone would therefore have insufficient prognostic power to be considered as a biomarker.

Data from the multi-patient gastric cancer cohort was taken to test/simulate what TUMERIC-solo data for biglycan would look like in putative individual metastatic gastric cancer patients with different pembrolizumab treatment outcomes (FIG. 18). It is thereby shown that biglycan cancer/stroma expression levels can be inferred by TUMERIC-solo for a given patient to determine whether pembrolizumab will be effective in the setting of metastatic gastric cancer.

The identification of biomarkers predictive of response to PD-L1 inhibition is shown by way of a further working example showing the joint TUMERIC analysis of a clinical trial cohort and treatment-naïve microsatellite instable (MSI)/microsatellite stable (MSS) tumours.

Discovery of robust predictive biomarkers of response to immune checkpoint inhibition (ICI) therapy is challenged by the scarcity of transcriptomic data available from tumours of responders and non-responders of ICI treatment. Since microsatellite instable (MSI) tumours often have strong clinical responses to ICI therapy, a joint TUMERIC analysis of an immune checkpoint inhibition (ICI) clinical trial cohort and a large cohort of treatment-naïve microsatellite instable (MSI)/microsatellite stable (MSS) tumours was performed. This joint analysis yielded 5 cancer and 6 stromal-compartment gene expression biomarkers robustly associated with both ICI response and MSI status across three different tumour types.

Microsatellite instablility is frequent in colorectal, gastric, and uterine endometrial carcinomas. A cohort of ˜1000 treatment-naïve tumours were assembled from these three tumour types in TCGA. Using TUMERIC, cancer and stromal-cell gene expression differences between microsatellite instable (MSI) and microsatellite stable (MSS) tumours that were present in all 3 tumour types were identified. Next, TUMERIC was used to analyse transcriptome data from a clinical trial of metastatic gastric cancer patients treated with a PD-L1 inhibitor (pembrolizumab; Nature Medicine. 2018, DOI: 10.1038/s41591-018-0101-z; the information disclosed in this study can also be found in the European Nucleotide Archive [ENA; part of the ELIXIR infrastructure of the EMBL-EBI, Wellcome Genome Campus, Hinxton, Cambridgeshire, CB101 SD, UK] under study number PRJEB25780). Briefly, patients were divided into groups based on their treatment response (complete/partial response (R); stable disease (SD); progressive disease (PD)), and TUMERIC was applied within each group of patients. Significant cancer and stromal cell gene expression differences were then identified between the complete/partial response (R) and the progressive disease (PD) groups. Finally, the biomarkers from the MSI/MSS and clinical trial data analysis were intersected, which yielded a final list of 6 stromal cell-associated biomarkers (IFNG, FASLG, CXCL13, ZNF683, IL2RA, and CD274/PD-L1) and 5 cancer cell-associated biomarkers (CPNE1, TTC19, OXCT1, ALDH6A1, and COX15). The compartment-specific gene expression changes of these biomarkers can be measured in individual patient tumours by applying TUMERIC-Solo, and the compartment-specific changes can then be used to predict response to ICI treatment. The data for the identified biomarker genes are summarized in FIGS. 24 to 34.

Treatments envision in the scope of this disclosure include, but are not limited to, cancer cell-targeting antibodies (for example, e.g. ADCs), therapeutic antibodies against, for example, cell surface receptors, as well as chemotherapeutic agents.

In another example, the method disclosed herein further comprising selecting genes or transcript isoforms for antibody based therapy and/or T-cell based therapy.

The advantages of the methods disclosed herein include that these methods are applicable to both frozen and formalin-fixed paraffin-embedded (FFPE) tissue samples, meaning that one can still undertake immunohistochemical staining and the like after analysis. Also, as illustrated in the data provided herein, the disclosed methods are capable of differentiating between cancer and stromal (any non-cancer) cell types and provide more information than bulk/average profiling. Also, while the currently disclosed method focusses on transcriptomic profiling, it would be possible to adapt the same to other types of “Omics” (for example, but not limited to epigenomics, proteomics and the like). As disclosed herein the current method is guided by parallel DNA sequencing and could also be performed with data from sectored RNA data alone (for example, with purity estimation based on RNA expression alone).

The method can also be applied to complementary approaches in studies of tumour microenvironment cell biology and antibody drug discovery in settings where bulk tumour biopsy data is either already abundant, or the only feasible data source. Furthermore, the insights gained from the method can be used to design in vitro assays and co-culture models that more accurately mimic the biology of the human tumour microenvironment.

Thus, it can be seen that the disclosed method has the potential to revolutionize the molecular data that can be extracted from individual bulk tumour samples. It is envisioned that using methodologies in accordance with the present embodiment will create a near-term future where the cost of sequencing drops >10-fold ($100 genome), meaning that the additional sequencing cost (˜5 fold higher) associated with the approach disclosed herein will become negligible compared to the overall administrative and handling overhead associated with sequencing as a service for bulk tumour samples. The ability to directly and unbiasedly profile cancer cells from bulk tumour samples should be of immediate interest to companies selling clinical sequencing as a service, precision oncology operations at cancer hospitals, and large pharmaceutical companies interested in development of companion biomarkers. The methodologies in accordance with the present embodiment can be used for any molecular activity (mRNA, epigenetic, protein expression) that can be co-extracted from the individual section and is ideally suited for analysis of mRNA expression, as DNA and RNA can effortlessly be co-extracted and analysed by next-generation sequencing.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Methods

### Tumour Data Sources

Twenty solid tumour types were analysed. These solid tumour types have the Cancer Genome Atlas (TCGA) acronyms BLCA (Bladder Urothelial Carcinoma), BRCA (Breast Invasive Carcinoma), CESC (Cervical Squamous Cell Carcinoma), CRC (Colon and Rectum Adenocarcinoma) (COAD (Colon adenocarcinoma) and READ (Rectum adenocarcinoma) combined), ESCA (Esophageal Carcinoma), GBM (Glioblastoma Multiforme), HNSC (Head and Neck Squamous Cell Carcinoma), KIRC (Kidney Renal Clear Cell Carcinoma), KIRP (Kidney Renal Papillary Cell Carcinoma), LGG (Brain Lower Grade Glioma), LIHC (Liver Hepatocellular Carcinoma), LUAD (Lung Adenocarcinoma), LUSC (Lung Squamous Cell Carcinoma), OV (Ovarian Serous Cystadenocarcinoma), PAAD (Pancreatic Adenocarcinoma), PRAD (Prostate Adenocarcinoma), SKCM (Skin Cutaneous Melanoma), STAD (Stomach Adenocarcinoma), THCA (Thyroid Carcinoma) and UCEC (Uterine Corpus Endometrial Carcinoma). Somatic mutation (SNV) and copy number variation (CNV) data for the twenty tumour types was obtained from the Broad Institute Firehose website (See data accession section below). Uniformly processed Cancer Genome Atlas RNA-sequencing (FPKM) data was obtained from the UCSC Xena server.

### Tumour Purity Estimation

Four different published methods for consensus tumour purity estimation were used. These are AbsCNseq, PurBayes, Ascat and ESTIMATE. AbsCNseq uses copy number alterations segmentation and single nucleotide variant (SNV) variant allele frequency (VAF) data of individual tumours. PurBayes utilizes SNV VAF data of diploid genes (inferred from copy number alterations data). Ascat purity estimation is based upon copy number alterations (single nucleotide polymorphism (SNP) array) data, where tumour ploidy and purity are co-estimated to identify allele specific copy number alterations. Pre-computed Ascat tumour purity estimates for the Cancer Genome Atlas cohort were obtained from the COSMIC website (See data accession section below). ESTIMATE uses mRNA expression signatures of known immune and stromal gene signatures to infer tumour purity, and tumour purity values were obtained by applying ESTIMATE to the Cancer Genome Atlas RNA-sequencing (log 2 FPKM [fragments per kilobase]) data. In order to derive consensus tumour purity estimates, missing data imputation was carried out, followed by quantile normalization separately for each cancer type. Some tumour purity values were missing because the algorithms failed to on certain input data instances. Additionally, some instances of very high (>98%) or low (<10%) purity estimates were observed, but such cases were usually only found by a single method for a given tumour and were therefore also assigned as missing data. Missing data was then imputed using an iterative Principal Component Analysis of the incomplete algorithm-vs-sample tumour purity matrix (using the missMDA R package).

Quantile normalization was used to further standardize the tumour purity distributions of different algorithms. Briefly, the tumour purity values are sorted for each algorithm, and a mean value is computed for each rank in these distributions. These mean values are substituted back into the individual purity distributions. Since ESTIMATE generated purity estimates with a large bias compared to the other three methods (generally 30-50% higher), only ESTIMATE purity values were used in the ranking step. The final TUMERIC consensus tumour purity estimate was obtained as the mean of these normalized purity values.

### Cancer-Stroma Gene Expression Deconvolution

It was assumed that tumours are comprised of cancer and stromal (any non-cancer) cells. Measured bulk tumour mRNA abundance was then determined by the sum of mRNA molecules derived from these two compartments. mRNA expression measured for a given gene in sample i can then be expressed as shown in Equation 3:

e_{tumor,i}=pᵢ×ē_{cancer}+(1−pᵢ)×ēₛₜᵣₒₘₐ (3)

Here pi denotes the cancer cell proportion (tumour purity), and ē_{cancer} and ēₛₜᵣₒₘₐ are average expression levels for the gene in the cancer and stromal compartment, respectively. Reference is also made to FIG. 21, which shows the underlying mathematical model disclosed herein. The simplifying assumption was made that these (non-negative) average compartment expression levels are constant across a set of tumours, which were estimated using non-negative least squares regression (SciPy library). RNA-sequencing fragments per kilobase (FPKM) data was log-transformed before deconvolution, log 2(X+1). It has been discussed whether gene expression deconvolution should be done using linear or log-transformed gene expression values. Firstly, it was observed that the relationship between tumour purity and bulk tumour gene expression was often heteroscedastic. Secondly, both transformations were evaluated, and while results where overall similar, it was found that the log-transformation provided improved separation between inferred cancer and stroma compartment gene expression for known stromal genes (data not shown). It was also found that the equation above (Equation 1) tended to overestimate stromal gene expression for genes with somatic copy number alterations (CNA) affecting gene expression in a subset of the samples (for example ERBB2 in HER2-positive breast tumours). Therefore, a modified approach was used for such genes. Genes were identified using the correlation between copy number alterations (CNA) and mRNA expression (comparing expression for samples with diploid and non-diploid copy number alterations, Mann-Whitney U-test, P<1 e-6, to account for multiple testing) in a given set of tumours, and then cancer and stromal compartment gene expression were estimated using a two-step approach. Stromal compartment mRNA expression was first inferred using the above approach using only samples having diploid copy number for the gene. The inferred mean stroma compartment expression, the measured mean tumour expression, and the mean purity of the tumour samples were then used to calculate the mean cancer compartment expression using above equation.

Deconvolution of iTRAQ Tumor Protein Expression Data

The iTRAQ data for BRCA (breast cancer) and ovarian cancer (OV) tumour types was obtained using CPTAC consortium data available at cBioPortal (www.cbioportal.org). The data was deconvoluted into cancer and stroma compartment expression similar to RNA-sequencing data described above.

### Ligand-Receptor Relative Crosstalk (RC) Score

To estimate the relative flow of signalling between cancer and stromal cell compartments, the Relative Crosstalk (RC) score was developed. Ligand-receptor (LR) complex activity is estimated using the product of gene expression inferred for the given compartments (linear scale). The RC score as calculated in Equation 4 then estimates the relative complex activity given all four possible directions of signalling and a normal tissue state, e.g. for cancer-cancer (CC) signalling:

$${RC}_{CC} = \frac{( {e}_{L , C} \times {e}_{R , C} )}{\begin{matrix}{e}_{L , C} \times {e}_{R , C} + {e}_{L , C} \times {e}_{R , S} + {e}_{L , S} \times {e}_{R , C} + \\ {e}_{L , S} \times {e}_{R , S} + {e}_{L , N} \times {e}_{R , N}\end{matrix}} \qquad ( 4 )$$

The normal term in the denominator is included to account for complex activity in normal tissue, and this term is calculated directly from the observed gene expression levels in matched normal tissue samples available for each tumour type in TCGA. It is noted that the Relative Crosstalk (RC) score is based on a number of simplifying assumptions, for example that there are no competition or saturation effects for individual ligand-receptor complexes, mRNA expression is a reasonable proxy for ligand and receptor concentration at the site of ligand-receptor-complex formation, that cancer and stromal cells are uniformly mixed in the tumour, and that all cancer and stromal cells have the same properties and gene expression profiles.

### Gene-Set Enrichment (GSEA) Analysis

To study genes differentially expressed between cancer and stromal cells, gene-set enrichment (GSEA) analysis was performed on pre-ranked analysis of genes sorted by differential expression (log fragments per kilobase) in cancer and stromal compartments. All hallmark gene signatures were analysed, and a false-discovery rate (FDR) cut-off of 0.25 was used to determine gene sets with differential enrichment.

### Immunohistochemistry (IHC) Quantification Analysis

In order to quantify cancer and stromal cells expression of genes, colour deconvolution of IHC images obtained from the Human Protein Atlas (proteinatlas.org) was performed using the ImageJ software package and standard protocols. Following manual selection and segmentation of cancer and stromal cells (without knowledge of antibody staining), colour intensities were measured with ImageJ, and DAB (target), hematoxylin (cells), and complementary components were estimated. Average antibody intensities were then estimated for the cancer and stromal compartment of a given slide. In summary, IHC images of various human tumour samples stained with antibodies for S100A6 and LDHB were obtained from the Human Protein Atlas and analysed with the ImageJ softwarr. Colour deconvolution of DAB and hematoxylin was performed using the protocol described by Ruifrok et al. First, two good quality images with clearly visible cancer and stroma cells were randomly selected. Next, the stroma and cancer cells of each IHC image were manually detected and segmented (using ROI manager) to stroma and cancer regions based on pathological features (cancer type, size, shape, arrangement of the cells and cell's nucleus) [3]. Pixel intensities were then calculated for the identified cancer and stroma regions based on the DAB vector (antibody). The fraction of each cancer/stroma region with DAB staining was estimated and an average cancer/stroma staining score was calculated according to Equation 5 (as shown below) for the entire slide:

log₂((mean_cancer_staining_fraction+1%)/(mean_stroma_staining_fraction+1%) (5)

A pseudocount of 1% was added to numerator and denominator to handle cases of zero cancer/stroma staining.

### Tables

TABLE 1
TCGA cancer types and samples used. See also FIG. 22.

| | | NO. | NO. |
|---|---|---|---|
| | | TUMOR | NORMAL |
| ID | TUMOR TYPE | SAMPLES | SAMPLES |
| BLCA | Bladder Urothelial Carcinoma | 407 | 19 |
| BRCA | Breast Invasive Carcinoma | 1082 | 113 |
| CESC | Cervical Squamous Cell Carcinoma | 304 | 3 |
| CRC | Colon and Rectum Adenocarcinoma | 372 | 51 |
| ESCA | Esophageal Carcinoma | 181 | 11 |
| GBM | Glioblastoma Multiforme | 163 | 5 |
| HNSC | Head and Neck Squamous Cell | 502 | 44 |
| | Carcinoma | | |
| KIRC | Kidney Renal Clear Cell Carcinoma | 382 | 72 |
| KIRP | Kidney Renal Papillary Cell | 286 | 32 |
| | Carcinoma | | |
| LGG | Brain Lower Grade Glioma | 522 | 5 |
| LIHC | Liver Hepatocellular Carcinoma | 369 | 50 |
| LUAD | Lung Adenocarcinoma | 513 | 59 |
| LUSC | Lung Squamous Cell Carcinoma | 485 | 50 |
| OV | Ovarian Serous Cystadenocarcinoma | 304 | NA |
| PAAD | Pancreatic Adenocarcinoma | 178 | 4 |
| PRAD | Prostate Adenocarcinoma | 494 | 52 |
| SKCM | Skin Cutaneous Melanoma | 468 | 1 |
| STAD | Stomach Adenocarcinoma | 412 | 34 |
| THCA | Thyroid Carcinoma | 501 | 59 |
| UCEC | Uterine Corpus Endometrial | 181 | 23 |
| | Carcinoma | | |

1. A method of predicting expression profiles of cancerous and non-cancerous cells, respectively, based on multiple sets of expression profiles, wherein each set of the multiple sets of expression profiles is obtained from tumor-derived samples comprising a mixture of cancerous and non-cancerous cells of one tumor type, wherein the method comprises the following steps:
a. determining tumor purity values for the one or more tumor-derived samples;
b. providing different sets of expression profiles, wherein the sets of expression profiles comprise combined expression data for multiple or all molecules expressed by cancerous and non-cancerous cells comprised in the one or more tumor-derived samples;
c. deconvoluting each combined expression data referred under b. by extrapolating expression profiles of the multiple or all molecules expressed in the different tumor samples with different tumor purity values to a tumor purity value at least substantially equal to 1 or 0; thereby predicting the expression profiles of the cancerous and non-cancerous cells respectively from the sets of expression profiles.

2. The method of claim 1, wherein the tumor-derived sample is obtained from a single subject.

3. The method of claim 2, wherein the tumor-derived sample is partitioned into 2 or more sections, and wherein one set of expression profiles is generated for each section.

4. The method of claim 1, wherein providing different sets of expression profile comprises use of existing data sets of expression profiles.

5. The method of claim 4, wherein the existing data sets of expression profiles are from TCGA and ICGC databases.

6. The method of claim 1, wherein the tumor type is selected from the group consisting of BLCA (Bladder Urothelial Carcinoma), BRCA (Breast Invasive Carcinoma), CESC (Cervical Squamous Cell Carcinoma), CRC (Colon and Rectum Adenocarcinoma) (COAD (Colon adenocarcinoma) and READ (Rectum adenocarcinoma) combined), ESCA (Esophageal Carcinoma), GBM (Glioblastoma Multiforme), HNSC (Head and Neck Squamous Cell Carcinoma), KIRC (Kidney Renal Clear Cell Carcinoma), KIRP (Kidney Renal Papillary Cell Carcinoma), LGG (Brain Lower Grade Glioma), LIHC (Liver Hepatocellular Carcinoma), LUAD (Lung Adenocarcinoma), LUSC (Lung Squamous Cell Carcinoma), OV (Ovarian Serous Cystadenocarcinoma), PAAD (Pancreatic Adenocarcinoma), PRAD (Prostate Adenocarcinoma), SKCM (Skin Cutaneous Melanoma), STAD (Stomach Adenocarcinoma), THCA (Thyroid Carcinoma), and UCEC (Uterine Corpus Endometrial Carcinoma).

7. The method of claim 1, wherein the expression profiles are selected from the group consisting of gene expression, RNA expression, epigenetic expression, protein expression, proteomic expression, and combinations thereof, for example, RNA and epigenetic expression, and RNA and protein expression.

8. The method of claim 1, wherein the method for determining tumor purity is selected from the group consisting of distribution of somatic DNA variant allele frequencies, somatic DNA copy number alteration amplitudes, germline B-allele frequencies, gene expression signatures or patterns, protein expression signatures or patterns, DNA methylation signatures or patterns, and combinations thereof.

9. The method of claim 8, wherein at least two, or at least three, or at least four, or at least five, or two, or three, or four, or five or all of the methods of claim 8 are used together to determine mean tumor purity.

10. The method of claim 1, wherein tumor purity value is a mean tumor purity value.

11. The method of claim 1, further comprising scoring molecules of step c. based on the level of up-regulation or down-regulation in cancer tissue versus stromal tissue; and/or scoring molecules of step c. based on the level of up-regulation or down-regulation in cancer tissue versus healthy tissue.

12. The method of claim 11, further comprising assigning the up- and down-regulated molecules to genes or transcript isoforms of known data sets of membrane associated proteins or receptors; and/or assigning the up- and down-regulated molecules to genes or transcript isoforms of known data sets of HLA-binding peptides and T-cell antigen binding peptides.

13. The method of claim 12, wherein the known data sets for assigning genes or transcript isoforms originates from Gene Ontology and/or TANTIGEN.

14. The method of claim 12, further comprising selecting genes or transcript isoforms for antibody based therapy and/or T-cell based therapy.

15. The method of claim 12, wherein the gene or transcript isoform is a membrane associated protein, membrane associated receptor, antigen peptide, target protein, peptide, and/or is targetable by an antibody.

16. The method of claim 1, wherein the molecules are selected from the group consisting of gene, DNA, RNA or protein molecules, or combinations thereof.
